(19) United States
(12) Patent Application Publication
(10) Pub. No.: US 2024/0041303 A1
(43) Pub. Date: Feb. 8, 2024

(54) STEERABLE ENDOSCOPES AND RELATED SYSTEMS AND METHODS

(71) Applicant: Malia Medical, LLC, Logan, UT (US)

(72) Inventors: Thomas William Winegar, Trumbull, CT (US); Ashley N. Holbrooks, New Haven, CT (US); Xiao WU, Orange, CT (US); Norman Joseph Labrecque, Trumbull, CT (US); Mark E. Prosachik, Farmington, CT (US)

(73) Assignee: Malia Medical, LLC, Logan, UT (US)

(21) Appl. No.: 18/382,440

(22) Filed: Oct. 20, 2023

Related U.S. Application Data
(63) Continuation of application No. PCT/US2022/025654, filed on Apr. 20, 2022.
(60) Provisional application No. 63/177,155, filed on Apr. 20, 2021.

(51) Int. Cl. A61B 1/005; A61B 1/01; A61B 1/018; A61B 1/00

(52) U.S. Cl. CPC A61B 1/0052; A61B 1/0057; A61B 1/01; A61B 1/018; A61B 1/00103

(57) ABSTRACT

An endoscope can include a handle, an actuator coupled to the handle, and an elongated shaft coupled to the handle. The shaft can include a deflectable distal portion, an imaging assembly at a distal end of the shaft, a tube that defines a lumen, and a guide positioned within the lumen of the tube. The guide can include a first groove that cooperates with the tube to define a first lumen and a second groove that cooperates with the tube to define a second lumen through which communication lines associated with the imaging assembly pass. The endoscope can further include a steering wire coupled to the deflectable distal portion of the shaft, extending through the first lumen and coupled to the actuator, and coupled with the actuator such that actuation of the actuator causes the steering wire to deflect the distal portion of the shaft.

### CROSS-REFERENCE TO RELATED APPLICATIONS

This application is a continuation of International Application No. PCT/US2022/025654, filed Apr. 20, 2022, titled STEERABLE ENDOSCOPES AND RELATED SYSTEMS AND METHODS, which claims priority to U.S. Provisional Patent Application No. 63/177,155, filed Apr. 20, 2021, titled STEERABLE ENDOSCOPES AND RELATED METHODS, the entire contents of each of which are hereby incorporated by reference herein.

### TECHNICAL FIELD

Certain embodiments described herein relate generally to endoscopes, and further embodiments relate more particularly to single-use endoscopes, such as hysteroscopes or cystoscopes, and related systems and methods.

### BACKGROUND

Endoscopes, including single-use or disposable varieties and some steerable versions, are well known. Known endoscopes suffer from a variety of drawbacks or limitations, however, and are not well-suited for every application. Embodiments disclosed herein ameliorate and/or avoid drawbacks or limitations of known endoscopes.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view of an embodiment of an endoscope;

FIG. 2A is a top plan view of the endoscope with a covering removed from a shaft thereof to facilitate viewing of an underlying tube, wherein the endoscope is shown in an undeflected state;

FIG. 2B is another top plan view of the endoscope with the covering removed, wherein the endoscope is shown in a deflected state;

FIG. 3A is a cross-sectional view of a distal portion of an insertion shaft of the endoscope with the covering removed, wherein the insertion shaft is shown in the undeflected state;

FIG. 3B is a cross-sectional view of the distal portion of the insertion shaft with the covering removed, wherein the shaft is shown in the deflected state;

FIG. 4 is a perspective view of an embodiment of the insertion shaft of the endoscope with a covering removed to facilitate viewing of an underlying tube;

FIG. 5 is a perspective cross-sectional view of a portion of an embodiment of an insert or guide configured to be positioned within the tube of an insertion shaft of an endoscope;

FIG. 6 is a perspective cross-sectional view of the insert positioned within the tube of an embodiment of a shaft of an endoscope;

FIG. 7A is a perspective view of an embodiment of an endoscope that includes a selectively attachable working channel accessory, wherein the accessory is shown in a detached state and in the process of being coupled to the endoscope;

FIG. 7B is another perspective view of the endoscope of FIG. 7A with the working channel accessory attached;

FIG. 8 is a side elevation view of a distal portion of an embodiment of a shaft portion of a working channel accessory;

FIG. 9A is a top plan view of an embodiment of an endoscope showing a handle with a top housing portion removed to facilitate viewing of an embodiment of an actuator;

FIG. 9B is a top plan view of another embodiment of an endoscope showing a handle with a top housing portion removed to facilitate viewing of another embodiment of an actuator;

FIG. 10 is a perspective view of an embodiment of an endoscope that includes a two-part handle having a reusable component and a disposable component, wherein the components are shown in a detached state;

FIG. 11 is a cross-sectional view of a proximal portion of the endoscope of FIG. 10 with the handle in an assembled state;

FIG. 12 is an end-on elevation view of an embodiment of a distal tip of an endoscope shaft;

FIG. 13 is an end-on elevation view of a distal end of the endoscope shaft of FIG. 12 with a working channel accessory coupled thereto;

FIG. 14 is an elevation view of an embodiment of an endoscopic system that includes two separate single-use endoscopes that are interchangeably, individually couplable with a reusable handle, the handle being configured to wirelessly communicate with a tablet device and selectively couplable with a charging cradle;

FIG. 15 is an elevation view of an embodiment of an endoscope that is compatible with one or more components of embodiments of the endoscopic system of FIG. 14, wherein a distal portion of an insertion shaft of the endoscope is omitted to permit an enlarged view;

FIG. 16A is a front perspective view of the endoscope of FIG. 15;

FIG. 16B is a rear perspective view of the endoscope of FIG. 15;

FIG. 17 is another front perspective view of the endoscope of FIG. 15 with a housing component removed to permit viewing of interior components;

FIG. 18A is a perspective view of an embodiment of an adapter that is compatible with the endoscope of FIG. 15;

FIG. 18B is a cross-sectional view of the adapter of FIG. 18A taken along the view line 18B-18B in FIG. 18A;

FIG. 19A is a cross-sectional view of an intermediate portion of the adapter of FIG. 18A coupled with a fluid port and an embodiment of a channeling tube;

FIG. 19B is a cross-sectional view of an intermediate portion of another embodiment of an adapter coupled with a fluid port and an embodiment of a channeling tube;

FIG. 20 is a cross-sectional view of a distal portion of the adapter of FIG. 18A coupled with a proximal end of the insertion shaft;

FIG. 21 is a cross-sectional view of a distal end of the insertion shaft, wherein a portion of a bending section of the insertion shaft is omitted to permit an enlarged view;

FIG. 22A is a perspective view of a distal end of a guide member that is compatible with the endoscope of FIG. 15;

FIG. 22B is an end-on view of the distal tip of the guide member of FIG. 22A;

FIG. 23 is a perspective view of a distal end of the insertion shaft of the endoscope of FIG. 15;

FIG. 24 is an elevation view of another embodiment of an endoscope that is compatible with one or more components of embodiments of the endoscopic system of FIG. 14, wherein a distal portion of an insertion shaft of the endoscope is omitted to permit an enlarged view;

FIG. 25A is a front perspective view of the endoscope of FIG. 24;

FIG. 25B is a rear perspective view of the endoscope of FIG. 24;

FIG. 26 is another front perspective view of the endoscope of FIG. 24 with a housing component removed to permit viewing of interior components;

FIG. 27A is a perspective view of an embodiment of an adapter that is compatible with the endoscope of FIG. 24;

FIG. 27B is a cross-sectional view of the adapter of FIG. 27A taken along the view line 27B-27B in FIG. 27A;

FIG. 28 is a cross-sectional view of the endoscope of FIG. 24 taken along the view line 28 in FIG. 25A;

FIG. 29 is a cross-sectional view of a distal portion of the adapter of FIG. 27A coupled with a proximal end of the insertion shaft;

FIG. 30 is a cross-sectional view of a distal end of the insertion shaft, wherein a portion of a bending section of the insertion shaft is omitted to permit an enlarged view;

FIG. 31A is a cross-sectional view of the insertion shaft of the endoscope of FIG. 24;

FIG. 31B is another cross-sectional view of the insertion shaft with certain components thereof shown in phantom;

FIG. 32 is a perspective view of a distal end of the insertion shaft of the endoscope of FIG. 24;

FIG. 33 is a front perspective view of an embodiment of a handle that is compatible with one or more components of embodiments of the endoscopic system of FIG. 14;

FIG. 34 is another front perspective view of the handle of FIG. 33 with a housing component removed to permit viewing of certain interior components;

FIG. 35 is a perspective view of an embodiment of an electrical connector that is compatible with the handle of FIG. 33;

FIG. 36 is an enlarged perspective view of an end of the handle of FIG. 33 that includes the electrical connector of FIG. 35 fluidically sealed within a housing, wherein a mechanical connector defined by the housing is also shown;

FIG. 37 is a perspective view of an embodiment of an embodiment of an inductive coil that is compatible with the handle of FIG. 33;

FIG. 38A is a front perspective view of an embodiment of a charging cradle that is compatible with embodiments of the handle of FIG. 33;

FIG. 38B is a rear perspective view of the charging cradle of FIG. 38A;

FIG. 39 is another front perspective view of the charging cradle of FIG. 39 with a housing component removed to permit viewing of certain interior components; and

FIG. 40 is an elevation view of the handle of FIG. 33 coupled with the charging cradle of FIG. 38A.

### DETAILED DESCRIPTION

Certain known steerable endoscopes include a shaft that is flexible over a significant length. This flexibility can be achieved, for example, by including many individual links in the shaft that are assembled together. This can result in high manufacturing costs. In other instances, a rigid shaft or tube can be laser cut, often hundreds of times, to create flexibility. This, too, can result in high manufacturing costs, such as may result from significant time in laser-cutting machines.

In accessing and conducting procedures on or in certain organs, however, steerability can be isolated to just a distal end of the shaft, rather than an entirety thereof. For example, in may be desirable for a proximal portion of the shaft to be rigid and for only a distal end of the shaft to be flexible and steerable. In some instances, such an arrangement can advantageously reduce manufacturing costs of the shaft. For example, in some instances, a hybrid shaft that is rigid in proximal regions thereof and is flexible and steerable at a distal end thereof can be used advantageously in accessing and/or treating the uterus and/or bladder.

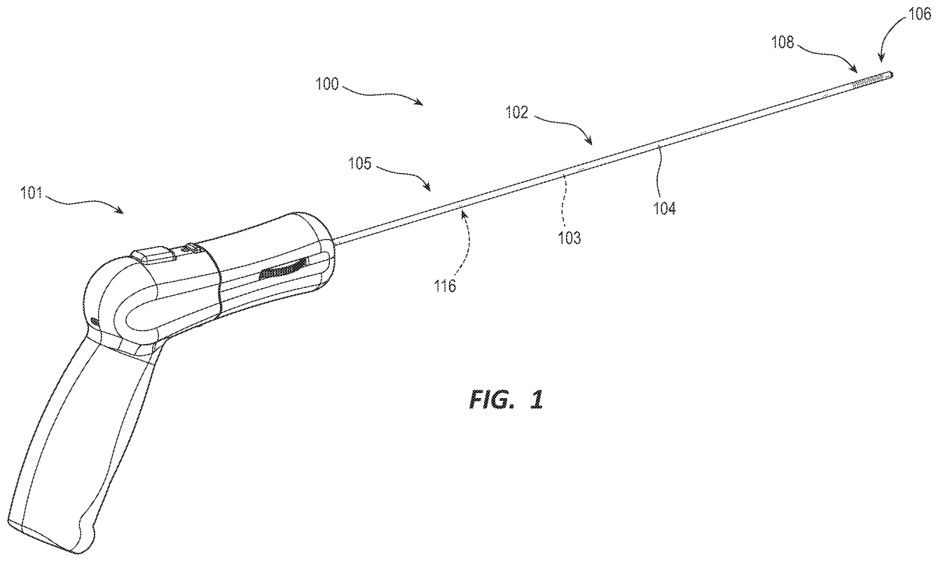

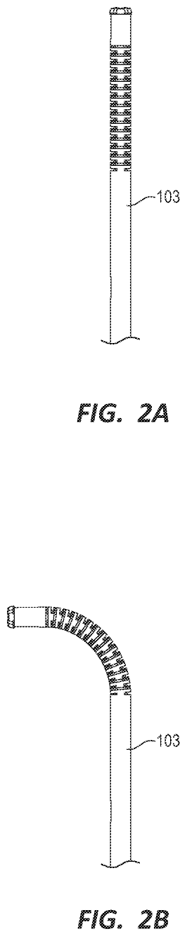

With reference to FIGS. 1-2B, certain embodiments of an endoscope 100 include a handle 101 and a shaft 102. The shaft 102 can include an elongated tube 103 and a covering 104 over the tube 103. The covering 104 is shown in FIG. 1, but is not shown in FIGS. 2A and 2B to permit better viewing of the tube 103. FIGS. 2A and 2B depict the endoscope 100 in two different operational configurations.

The endoscope may be a hysteroscope or a cystoscope, or may function in both capacities as a hysteroscope/cystoscope. The shaft 102 can include a rigid proximal portion 105 and a deflectable or steerable distal end 106. In some embodiments, the distal end 106 may be deflectable in a single direction. In other embodiments, the distal end 106 may be deflectable in multiple directions. For example, the distal end 106 may be deflectable in two directions, such as within a single plane, or, in other embodiments, may be deflectable in four directions, such as within two different planes.

The distal end 106 of the shaft 102 can include a bending section or bending region 108. In some embodiments, the tube 103 that extends continuously through both the proximal portion 105 and the distal end 106. The tube 103 can be rigid in the proximal region 105 and flexible in the bending region 108. At the bending region 108, the tube 103 can include a series of laser cuts that render the bending region 108 flexible so as to permit the distal end 106 of the shaft 102 to deflect relative to the proximal portion 105, or stated otherwise, so as to render the distal end 106 steerable within a patient.

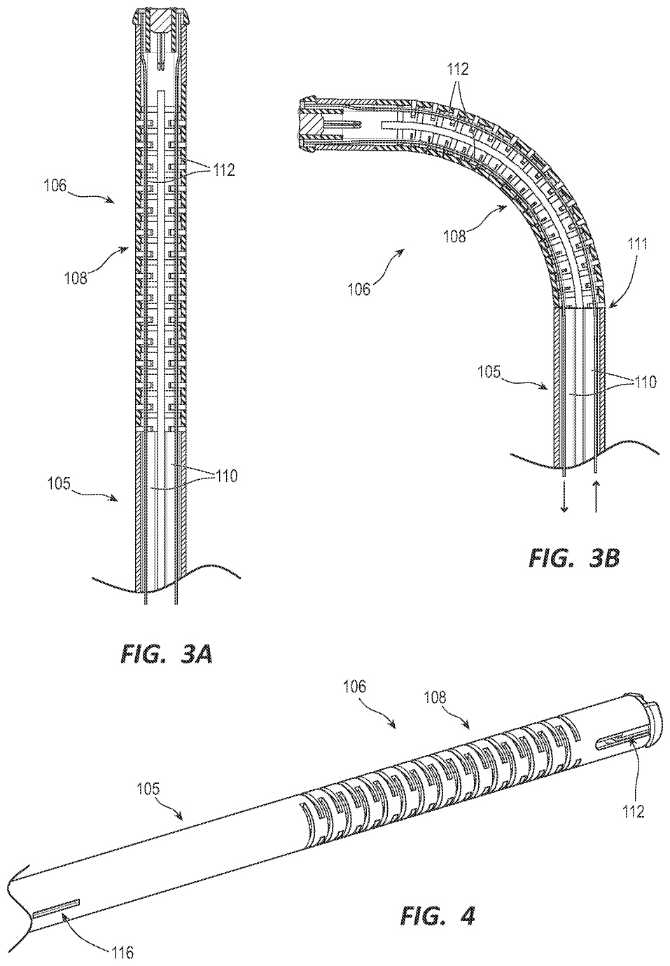

With reference to FIGS. 3A and 3B, in certain embodiments, the shaft 102 includes a plurality of guide tubes 110 that extend along at least a length thereof. The guide tubes 110 can retain steering wires 112 therein. The steering wires 112 can be coupled at their proximal ends to any suitable actuator and can be fixedly secured to the distal end 106 (e.g., at or near a distal tip of the shaft 102), such that actuation of the actuator can effectuate bending of the distal end 102. The guide tubes 110 can keep the steering wires 112 separated along the length of the shaft 102 and appropriately oriented to achieve deflection in two opposite directions. The illustrated embodiment includes a single pair of guide tubes 110 and steering wires 112 that are diametrically opposed within the shaft 102. In other embodiments, the shaft 102 can include two pairs of guide tubes 110 and steering wires 112, and in further embodiments, each pair is diametrically opposed, and in still further embodiments, the pairs are offset by 90 degrees such that the distal end 106 can be moved in two opposite directions along each of two different planes that are orthogonal to one another.

In some embodiments, the guide tubes 110 include Bowden cables, which can include tightly wound coils. In other embodiments, the guide tubes 110 may be formed in a more economical manner. For example, Bowden cables can be relatively expensive to manufacture due to their inclusion of wound coils. Bowden cables are often used in fully flexible endoscopes, where it is important to maintain at least some measure of flexibility along a full length of a shaft.

In some embodiments, rather than Bowden cables, the guide tubes 110 can be formed of rigid tubing of any suitable variety. In various embodiments, guide tubes 110 can include tubes formed of metal (e.g., stainless steel) or rigid plastic. For example, due to the rigidity of the proximal portion 105 of the shaft 102, the guide tubes 110 likewise can be rigid within the proximal portion 105. Each guide tube 110 can define a lumen or channel 111 through which a steering wire 112 passes.

In the illustrated embodiment, the guide tubes 110 are rigid and do not extend distally beyond a distal end of the proximal portion 105. In particular, in some embodiments, the guide tubes 110 may be rigid along their full length. The guide tubes 110 can terminate just proximal to the flexible bending region 108. In some instances, solid and/or rigid guide tubes as just discussed can reduce manufacturing costs.

In other embodiments, the guide tubes 110 may extend distally beyond a distal end of the proximal portion 105. For example, the guide tubes 110 may extend through at least a portion of the flexible bending region 108. In order to maintain flexibility in the bending region 108, the portion of a guide tube 110 that extends through the bending region 108 may be flexible. For example, in some embodiments, each guide tube 110 includes laser cuts that render the guide tube 110 at least as flexible as the bending region 108. The laser cuts may be positioned at along at least a length of the guide tube 110 that corresponds to the bending region 108.

In some embodiments, the guide tubes 110 are fixedly secured to the tube 103 in any suitable manner. For example, in some embodiments, the tube 103 and the guide tubes 110 are metallic and are welded together. In particular, the guide tubes 110 can be welded so as to run along an interior surface of the tube 103. In some instances, the tube 103 includes a plurality of windows or openings 116 at or through which the welding can be achieved. One opening 116 is depicted in FIG. 4. Another opening 116 (not visible) is positioned at an opposite side of the tube 103. Additional openings 116 can be positioned along a length of the tube 103. In other or further embodiments, adhesive may be used. For example, in some embodiments, the guide tubes 110 are polymeric and are adhered, rather than welded, to the tube 103.

With reference again to FIG. 1, the covering 104 can comprise any suitable material, such as polymeric sleeve of one or more layers. In some embodiments, the covering 104 comprises a heat shrink tubing of any suitable variety, durometer, and/or wall thickness. The covering 104 can cover and seal the openings 116 and/or laser cuts in the tube 103, such that fluid can be conducted through the tube 103 without leaking from the shaft 102 and/or between the tube 103 and the covering 104.

In some instances, it can be challenging to attach long lengths of, e.g., hypotubing to the tube 103 to serve as guide tubes 110. For example, in some instances the tubing may not be perfectly straight, so intimate contact for forming a weld joint or adhesive contact between the tubing and the tube 103 may not be readily attained. In some instances, fixturing may be used to maintain proper relative positioning of the guide tubes 110 relative to the tube 103 during manufacture.

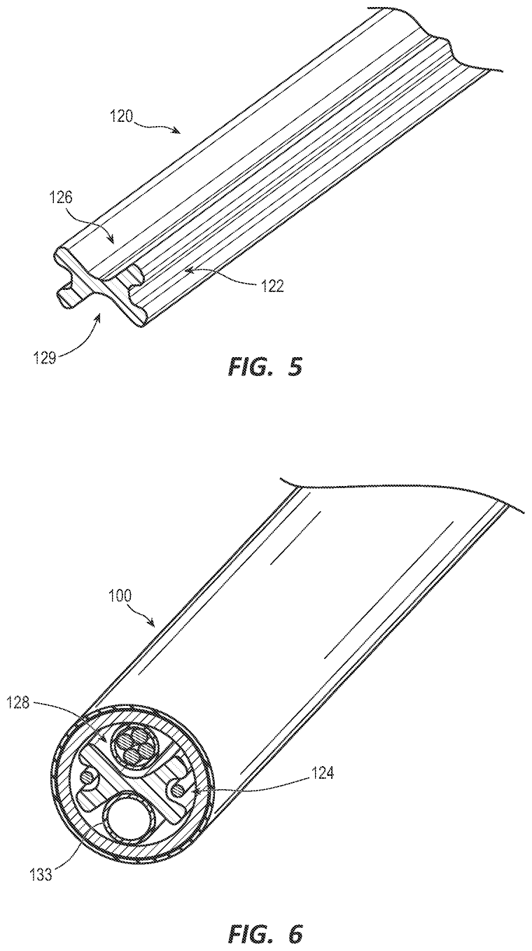

With reference to FIGS. 5 and 6, in some embodiments, an elongated guiding insert or guide 120 may be used in place of the guide tubes 110. The guide 120 and guide tubes 110 each may be referred to herein as a guide member. The guide member 120 can be positioned within the tube 103. The guide member 120 can define one or more longitudinal grooves or channels 122. The channels 122 can cooperate with an inner surface of the tube 103 to define distinct chambers or lumens 124 through which the control wires 112 can pass. In some instances, the guide member 120 can define one or more grooves or channels 126 for other purposes. For example, in the illustrated embodiment, a channel 126 cooperates with the inner surface of the tube 103 to define a lumen 128 through which one or more power and/or communication lines can pass, such as control and/or powering wires for a camera and/or a lighting source (e.g., one or more LEDs). A further channel 129 cooperates with the inner surface of the tube 103 to define a lumen 131 through which a channeling tube 133 extends.

In the illustrated embodiment, the guide member 120 defines a substantially X-shaped profile. Other shapes are contemplated and may result in more or fewer lumens through the tube 103. For example, in some embodiments, the guide member 120 includes a five-armed or a six-armed star shape to provide sufficient lumens for four control wires and a wire bundle.

In some embodiments, the guide member 120 is formed as an extruded polymeric material. Extruded plastic components can be relatively inexpensive, and may reduce manufacturing costs.

In some instances, the guide member 120 is sized such that friction between the guide member 120 and the tube 103 can hold the guide member 120 in place axially. Other or further attachment mechanisms are contemplated, such as, for example, adhesives. In other or further embodiments, a proximal end of the guide member 120 can be entrapped between separate portions of the handle 101 that are affixed together to assemble the handle 101 (e.g., between opposing handle halves). In some embodiments, the guide member 120 can be welded to the sidewall of the tube 103, as discussed elsewhere herein, which can reinforce the tube 103 and/or provide rigidity thereto.

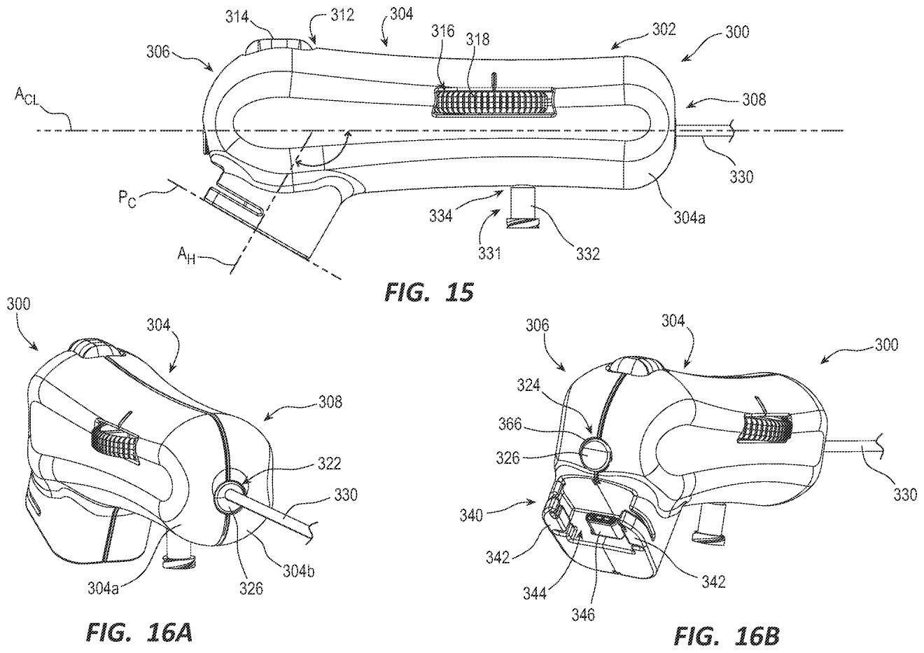

In still other or further embodiments, the guide member 120 may be formed of metal, and in still further embodiments, the guide member 120 may be welded to the tube 103 at intervals along the length of the tube 103, such as previously described above with respect to the guide tubes 110. For example, in some instances, the tube 103 can include openings 116 through which welding of the guide member 120 to the tube 103 can be achieved. A plurality of openings 116 can be positioned along a length of the tube 103. In some instances, the openings 116 may be present along four separate lines extending longitudinally along the length of the tube 103, such that each of the four longitudinal ribs of the embodiment of the guide member 120 depicted in FIG. 15 can be accessed and welded to the tube 103. Each longitudinally extending line along which one or more openings 116 may be located may correspond with an angular position of each rib of the guide member 120. Thus, the angular spacing between adjacent lines of openings 116 may correspond with the angular spacing between adjacent ribs or arms of the guide member 120.

By way of example, FIG. 1 shows one line of openings 116. And, as previously discussed, another line of four openings 116 can be angularly spaced from the first line of openings 116 by 180 degrees, or stated another way, can be located on the opposite side of the tube 103 so as not to be visible in the view depicted in FIG. 1. Such an arrangement may be present for guide tubes 110 that are angularly spaced apart by 180 degrees, such that the steering wires 112 that extend through the guide tubes 110 are at opposite sides of the tube 103. In similar fashion, four lines of any suitable number of openings 116 can extend along the tube 103 to provide welding access to each rib or arm of the guide member 120. In the illustrated embodiment, two opposite sets of adjacent ribs are each angularly spaced from each other by approximately 60 degrees, whereas two other opposite sets of adjacent ribs are each angularly spaced from each other by approximately 120 degrees. In like manner, adjacent lines of openings may be spaced from each other by 60, 120, 60, and 120 degrees around a periphery of the tube 103.

In some embodiments, the guide member 120 can be formed with relatively thin walls and/or can otherwise be relatively flexible. For example, even relatively high durometer materials may be extruded with thin walls such that the guide member 120 is highly or extremely flexible. Accordingly, in some embodiments, the guide member 120 may suitably extend through at least a portion of the bending region 108 without hindering operation thereof. In still other embodiments, endoscopes that are flexible even the proximal region 105 can employ the guide member 120.

In some embodiments, the guide member 120 may be positioned such that a distal end thereof is at or proximal to a proximal end of the bending region 108. For example, in some embodiments, a distal end of the guide member 120 may be restrained within the tube 103 to a position that is proximal to the distal region, similar to certain embodiments of the guide tubes 110 previously described.

If endoscopy is desired to be performed on an awake patient, for instance a hysteroscopy or cystoscopy performed in a doctor's office or clinic, minimizing the size of the cannula can be desirable. For instance, the dilation of the cervix due to the passage of a hysteroscope can be painful. If the size of the scope can be reduced, the pain associated with dilation due to the hysteroscope can likewise be reduced.

Some endoscopes, such as hysteroscopes, include a working channel through which instruments can be passed. The presence of a working channel can result in a relatively large outer diameter of the endoscope.

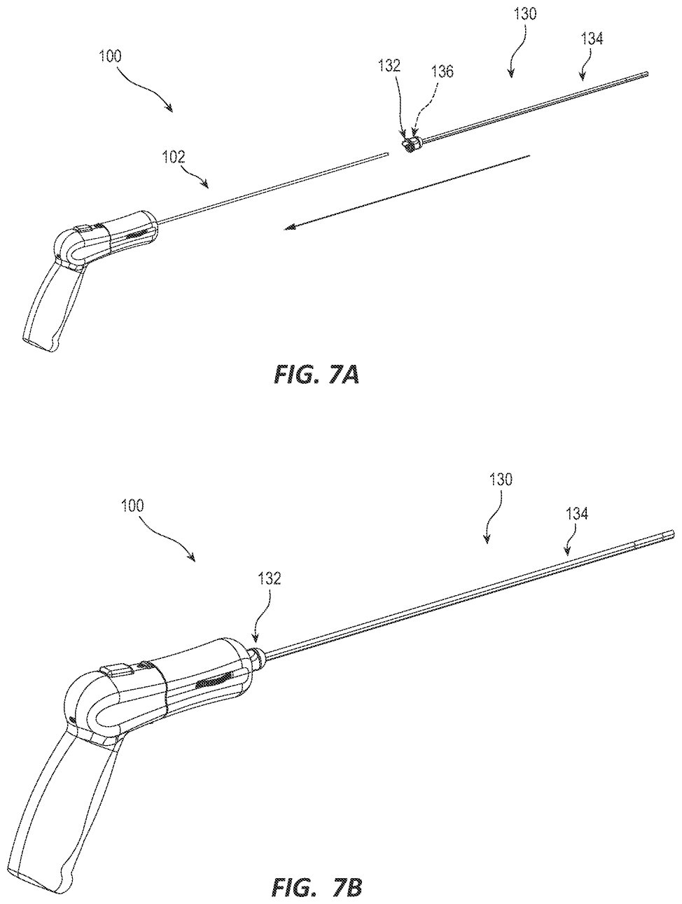

With reference to FIGS. 7A and 7B, in some embodiments, the endoscope 100 can have a shaft 102 that is devoid of a working channel. In further embodiments, the endoscope 100 can include a separate working channel accessory 130 that can be selectively attached to and removed from the shaft 102. The working channel accessory 130 may be used only when necessary or desired, which can advantageously permit the shaft 102 to have a reduced outer diameter for various (e.g., a majority of) insertions.

The working channel accessory 130 can include a connector 132 and an elongated shaft 134 attached to the connector 132 and extending distally therefrom. The shaft 134 can include at least one lumen sized to receive the shaft 102 of the endoscope 100 therein (see FIG. 13). In some embodiments, the shaft 102 further includes a separate lumen that functions as a working channel through which one or more instruments may be delivered. In other embodiments, the shaft 102 includes a single lumen that is sufficiently large to accept the shaft 102 therein and further define a working channel through which one or more instruments may be delivered.

In some embodiments, the working channel accessory 130 includes a valve 136 that is configured to permit an elongated instrument to pass therethrough into the working channel of the shaft 134 and provide a complete or partial seal about the elongated instrument. Any suitable valve mechanism is contemplated. The valve can advantageously allow the passage of the desired tool to perform the desired procedure through the working channel. The valve can stop at least the majority of fluid present from leaking back through the working channel, the connector 132, and/or the handle 101. In some embodiments, the valve is incorporated into the connector 132. In some instances, the valve can advantageously provide fluid management of the endoscope 100 without expensive fluid management capital equipment associated with certain endoscopes, such as endoscopes that may be used in the operating room, rather than clinic- or office-based products.

Again, reducing the overall size of instrumentation that is advanced into the patient can reduce pain associated with such insertion. To this end, it can be advantageous to reduce an outer diameter of the accessory 130. In some embodiments, a wall thickness of the channel or channels defined by the shaft 134 can be relatively thin. In certain embodiments, it is desirable to have the shaft 134 of the accessory 130 deflect in unison with deflection of the steerable cannula shaft 102. In some embodiments, the sidewall(s) of the shaft 134 are formed of a flexible material, which can assist with desired passive movement or bending of the shaft 134 as the shaft 102 is deflected. In various embodiments, and depending on the material used for the shaft 134, thin, flexible walls can be predisposed to stretch rather than deflect due to underlying movement of the shaft 102.

With reference to FIG. 8, in some embodiments, a stiff member 138 is included distal to a flexible, thin walled section of the shaft 134. The stiff member 138 can, for example, be a metal or plastic ring. In other or further embodiments, the stiff member 138 can include a section that has a higher durometer than a proximal portion of the shaft 134. For example, in some embodiments, the shaft 134 can include a coextrusion of the proximal portion of the shaft 134 and the stiff member 138. In certain embodiments, when the steerable cannula shaft 102 deflects, the stiff member 138 at the distal end moves with the shaft. Therefore, the flexible section does not see a force that would act to stretch it.

For example, in some embodiments in which the stiff member 138 is omitted, certain features that make the flexible region of shaft 134 flexible (e.g., low durometer or thin walls) could also make the distal opening of shaft 134 overly compliant and likely to stretch. In this scenario, when the distal tip of cannula shaft 102 deflects, the distal opening of shaft 134 elongates and/or enlarges, such that a tool passed through accessory 130 would not be pointed in the same direction as cannula shaft 102.

In contrast, the stiff member 138 can cause the distal opening of shaft 134 to move with the tip of cannula shaft 102 without elongation or enlargement. Stated otherwise, the stiff member 138 can maintain a preset configuration, even upon application of force thereto by the shaft 102 as the shaft 102 is deflected. The flexible portion of shaft 134 (see FIG. 8) would at least partially align, and in further instances, substantially align, with the bending region of the shaft 102. When shaft 102 deflects, the shaft 102 exerts a force on the distal region of shaft 134. Given that the force to bend the flexible portion of shaft 134 is less than the force required to elongate and/or enlarge the stiff member 138, the distal opening of shaft 134 will deflect along with shaft 102. In this way, a tool passed through accessory 130 can be advanced through the distal opening of the shaft 134 so as to remain aligned with (e.g., extend substantially parallel to) a distal end of the cannula shaft 102.

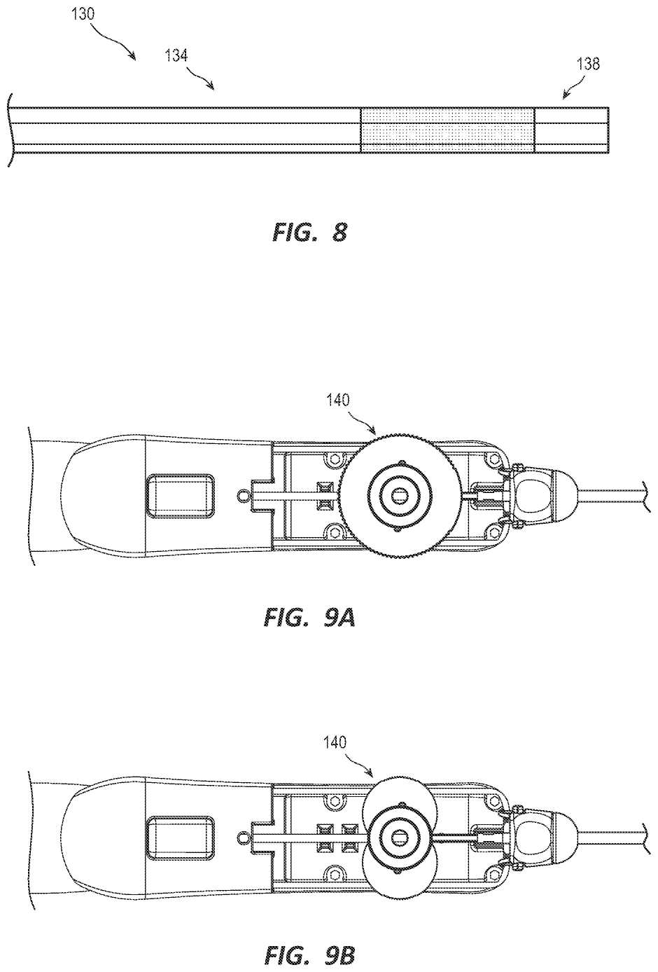

In some embodiments, it can be desirable for an actuator 140 that is coupled with the steering wires 112 to be actuatable via an index finger of a user. For example, such an arrangement can be ergonomically preferable. With reference to FIG. 9A, in some embodiments, the actuator 140 can comprise a steering wheel that is positioned relatively distally relative to the handle 101. With reference to FIG. 9B, in some embodiments, the actuator 140 comprises a dog bone or handlebar arrangement.

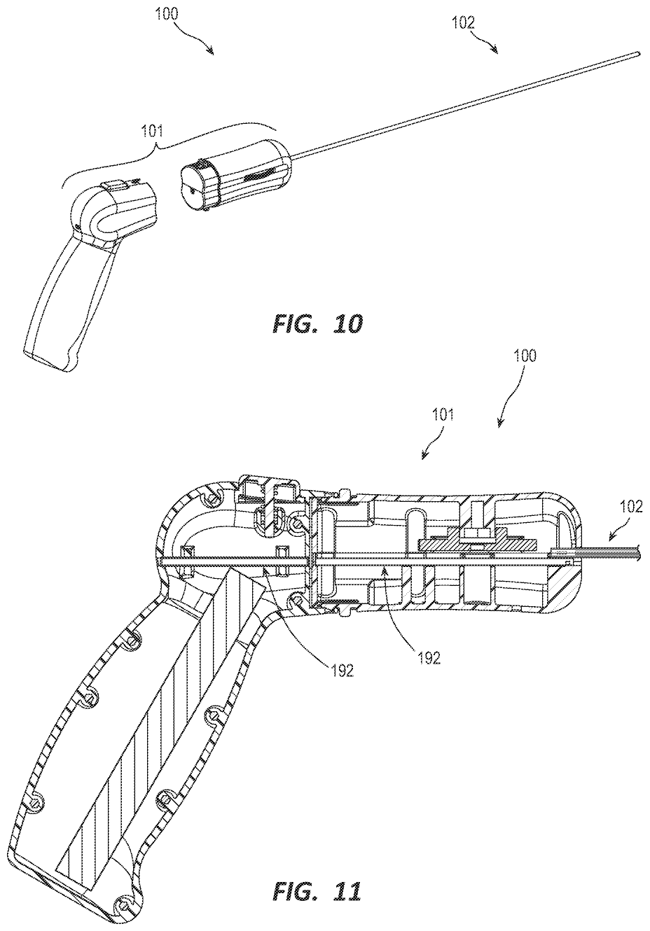

With reference to FIG. 10, in some instances, ergonomics of the endoscope 100 can be enhanced by reducing weight of the endoscope 100. In some embodiments, weigh reduction can be achieved by omitting an image processor or image processing hardware, a display, and/or other components from the endoscope 100. In some embodiments, the endoscope 100 can include a cable that communicates with a remotely positioned processor and display. In other embodiments, the endoscope 100 is configured to communicate with a processor and/or display remotely. Any suitable wireless technology is contemplated (Wi-Fi, Bluetooth, Bluetooth Low Energy (BLE), ZigBee, WiMAX, etc.) or similar technology.

In some embodiments, the handle 101 can be selectively separable to permit the wireless circuit to be integrated into a reusable portion of the handle 101, while a detachable portion of the handle 101 and the shaft 102 are disposable. The reusable and disposable sections can have any suitable mechanism for selective attachment/detachment. In some embodiments, a pistol or handle shape of the handle 101 can be ergonomically advantageous. In certain of such embodiments, it can be desirable to orient the connection mechanism such that the disposable portion is completely or nearly linear. This can reduce packaging waste, in some instances. In the embodiment illustrated in FIGS. 1-10, the disposable portion of the handle 101 includes the actuator 140 for controlling deflection of the shaft 102.

In some instances, it can be desirable for the reusable portion of the handle 101 to be resistant to fluid ingress, and therefore submersible. This can allow ease of reprocessing, for instance, by soaking in any suitable disinfecting agent, a such as Cidex. This can be accomplished by hermetically sealing the perimeter of the reusable portion, and using an appropriate electrical connector such as, for example, an IPX8 or greater rated USB-C connector.

With reference to FIG. 11, the handle 101 can define a lumen or channel that may be referred to as a working channel extension 192 through which instruments can be inserted into the working channel 190 when the working channel accessory 130 is coupled to, e.g., the handle 101. Each of the reusable and disposable portions of the handle 101 can define a separate portion of the working channel extension 192, and these portions can be aligned when the separable components are in a coupled state. The working channel extension 192 can be laterally offset from the insertion shaft 102. For example, in the illustrated embodiment, the working channel extension 192 defines a longitudinal axis that runs parallel to a longitudinal axis defined by the insertion shaft 102. Stated in yet another way, a lumen of the working channel extension 192 may be oriented such that a proximal projection thereof extends alongside an exterior surface of the insertion shaft 102.

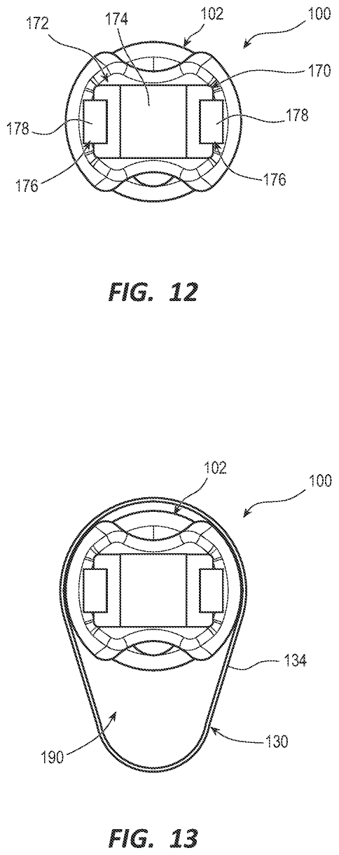

With reference to FIG. 12, in certain embodiments, a tip of the endoscope 100 can include an imaging system 170, which may also be referred to as an imaging assembly. The imaging assembly 170 can include an image-gathering system 172, of which at least a portion is positioned so as to directly receive imaging data from beyond a distal tip of the shaft 102 when the shaft 102 is inserted into the patient. Stated otherwise, at least a portion of the image-gathering system 172 can be oriented relative to the shaft 102 so as to observe a region distal to the distal tip of the shaft 102. In the illustrated embodiment, the image-gathering system 170 includes an imaging device 174 positioned at (e.g., embedded within) the distal tip.

Any suitable imaging device 174 is contemplated. For example, in the illustrated embodiment, the imaging device 174 includes an image sensor of any suitable variety, including those presently available and those yet to be devised. The image sensor may also be referred to as an imaging sensor or as an imager. The image sensor can, for example, comprise any suitable sensor that detects information used to make an image, which image can be a visual representation of the physical region detected by the sensor. Illustrative examples of suitable image sensors include analog and/or digital varieties of one or more of camera or video sensors, such as, for example, semiconductor charge-coupled devices or active pixel sensors of any suitable variety (e.g., complementary metal-oxide-semiconductor [CMOS], N-type metal-oxide-semiconductor [NMOS or Live MOS]). In other or further applications, suitable sensors may include thermal imaging sensors (e.g., infrared sensors), ultrasound sensors (e.g., ultrasonic transducers), etc. In some embodiments, the image sensor may be configured to convert an observed or detected phenomenon (e.g., light, infrared radiation, sound) into an analog or digital electrical signal representative of the observed phenomenon. In other embodiments, the image sensor may be configured to capture and/or directly transport the observed phenomenon. For example, in some embodiments, the image sensor may comprise an input end of a waveguide, such as an optical fiber or optical fiber bundle, which may capture and transport one or more signals away from the distal end of the sheath. In various embodiments, the image sensor can include one or more lenses or lens systems.

In the illustrated embodiment, the imaging device or image sensor 174 comprises a camera sensor configured to convert visible light into electrical signals that are representative of the detected light for transport through the endoscope 100. In some embodiments, the image sensor 174 comprises a CMOS camera, which in further embodiments, may comprise any suitable resolution. The camera may have a wide field of view. In various embodiments, the image sensor 174 may be referred to as a camera.

The image sensor 174 can be electrically coupled with one or more communication lines via which the electrical signals are transported through or along the sheath to electrical componentry within the two-part handle 101. The one or more communication lines may extend through at least a portion of the shaft 102, as previously discussed.

The imaging assembly 170 can further include a lighting assembly 176 that is positioned at (e.g., is embedded within) the distal tip. In the illustrated embodiment, the lighting assembly 176 includes a pair of cool LEDs or LED assemblies 178 at opposite sides of the image sensor 174. In some embodiments, operational controls of the LEDs are configured to auto-adjust an amount of light provided by the LEDs, such as to optimize the image quality obtainable by the image sensor 174.

Wiring for the imaging system 170 can extend through the shaft 102 and terminate in the disposable portion of the handle 101. Any suitable terminal(s) and/or connectors can be used to interface and establish communication (e.g., electrical communication) between the reusable and disposable portions of the handle 101 when these separate portions are physically connected together. The reusable portion can be used to provide power to the imaging system 170 and to establish and sustain communication between a control unit, viewing monitor, etc., and the imaging system 170. Any suitable physical coupling or connection interface is contemplated to selectively join the separable portions of the handle 101.

In some embodiments, a practitioner can use the endoscope 100 without the working channel accessory 130. For example, the practitioner may desire to use the endoscope 100 only for viewing and/or diagnosing purposes. A relatively smaller diameter of the endoscope 100 used without the working channel accessory 130 can reduce discomfort to a patient during use of the endoscope.

In other instances, it may be desirable to use the working channel accessory 130 with the endoscope 100 so as to provide access for one or more instruments, which may be inserted distally beyond the distal tip of the insertion shaft 102 to perform one of more procedures within the patient. With reference to FIG. 13, the working channel accessory 130 can include a working channel 190. In some embodiments, the working channel 190 can have an exit port in close proximity to the imaging system 170 when the working channel accessory 130 is coupled with the endoscope 100. A lumen of the working channel 190 can be defined by the inner surface of the shaft 134 and an outer surface of the insertion shaft 102.

With reference again to FIG. 11, when the working channel accessory 130 is coupled to the fully assembled handle 101, the working channel extension 192 aligns with the working channel 190 defined by the working channel accessory 130. Instruments thus may be passed through the working channel extension 192 and the working channel 190. The working channel extension 192, which extends through a full length of the handle 101, and the working channel 190 may be referred to collectively as the working channel of the fully assembled device.

As previously discussed, certain embodiments disclosed herein include an insertion shaft having a proximal portion that is rigid or stiff. In other embodiments, the proximal portion can be flexible.

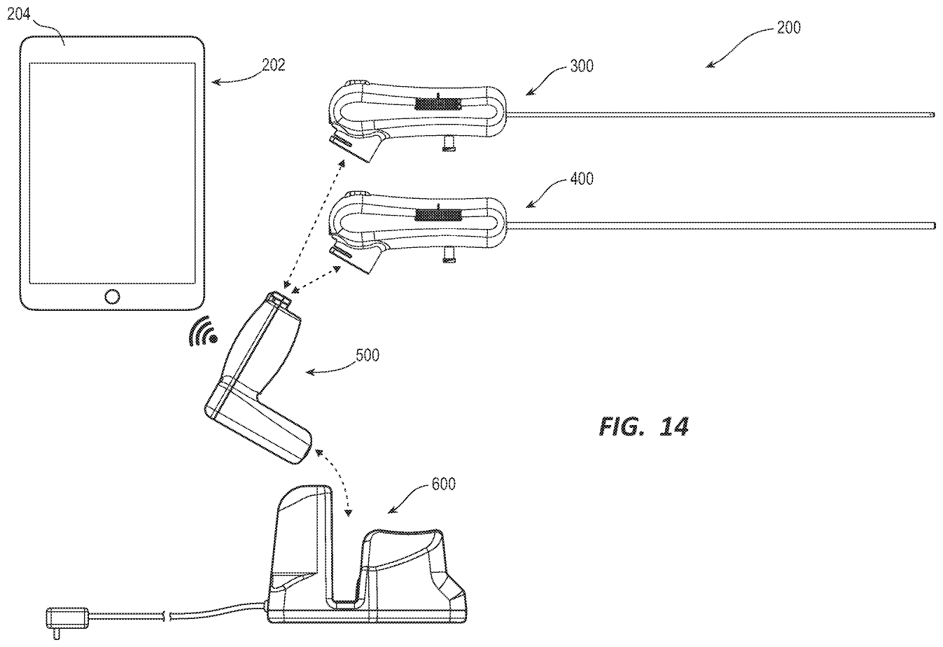

FIG. 14 depicts an embodiment of an endoscopic system 200 that includes two separate endoscopes 300, 400 that are interchangeably, individually couplable with a handle 500. In the illustrated embodiment, the system further includes a charging cradle 600 configured to couple with the handle 500 to charge a rechargeable battery positioned within the handle 500. In other embodiments, the handle 500 may be directly couplable with a power source (for example, the handle 500 may be of a corded variety, rather than untethered as shown).

In certain embodiments, the endoscopes 300, 400 are each of a single-use or disposable variety. The handle 500 can be reusable and, as further discussed below, can be fluid-tight so as to be capable of being cleaned or disinfected by submersion in a cleaning solution.

The handle 500 can be configured to communicate with a remotely positioned display unit 202 of any suitable variety. In the illustrated embodiment, the display unit 202 is a tablet 204 computing device. Any other suitable display unit and/or computing device is contemplated. In the illustrated embodiment, the handle 500 is configured to communicate wirelessly with the tablet 204, such as via any of the wireless communication protocols previously discussed (e.g., WiFi). In other embodiments, the handle 500 may instead include a cable for wired communication with the display unit 202.

As further discussed below, the endoscope 300 can be particularly well-suited for diagnostic uses that do not require use of a separate instrument within the patient. The endoscope 300 can be devoid of an instrument channel, which can permit a lower profile insertion shaft and thus reduce discomfort of a procedure. The endoscope 400, in contrast, can include an insertion channel through which one or more elongated instruments may be advanced into the patient (e.g., individually). A user thus may be able to select which endoscope 300, 400 is appropriate for a given procedure and couple either device with the handle 500. As further discussed below, the endoscopes 300, 400 may be interchangeably usable with the handle 500. In further instances, various features and/or components of the endoscopes 300, 400 may be identical to each other, which may facilitate and/or reduce the cost of manufacture of the endoscopes 300, 400. One or more of the foregoing advantages and/or other advantages of the system 200 will be apparent from the disclosure that follows.

With reference to FIGS. 15-16B, the endoscope 300 can include a handle 302, which may also be referred to as a body, base unit, control unit, controller, etc. The handle 302 can include a housing 304 that generally defines the overall shape and outer structure of the handle 302. The disposable handle 302 and the reusable handle 500 may collectively be referred to as a two-part handle, similar to the two-part handle 101 discussed above. For clarity in the disclosure, references hereafter to a “handle” generally will be directed to the reusable handle 500, whereas references to the disposable handle 302, as such, will be minimized, in favor of instead referring to the disposable handle 302 as “the body 302,” or in favor of instead referring to the housing 304 of the endoscope 300.

The housing 304 of the body 302 can be elongated in a longitudinal direction, or along a central longitudinal axis A_{CL} of the endoscope 300. Stated otherwise, the housing 304 can be elongated between a proximal end 306 and a distal end 308 of the body 302. The proximal and distal ends 306, 308 may also be referred to as the proximal and distal ends 306, 308 of the housing 304.

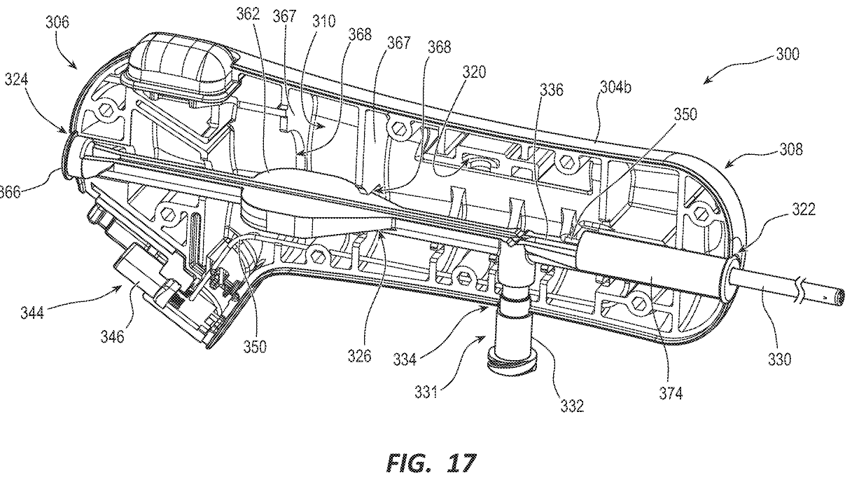

The housing 304 can include first and second housing elements or housing components 304a, 304b that can be joined together in any suitable fashion. The housing components 304a, 304b can define and enclose an internal or interior cavity 310 (see FIG. 17).

The housing 304 can define an opening 312 through which a camera actuator 314 can extend or otherwise be accessible by a user. In the illustrated embodiment, the camera actuator 314 can be pressed to take a still picture, commence a video recording, or end a video recording. For example, in some embodiments, the actuator 314 may be pressed relatively briefly to take a photo. A longer press and release will start a video recording, and a subsequent press (e.g., of any duration) will terminate the video recording upon release of the actuator 314. Any other suitable arrangement is contemplated.

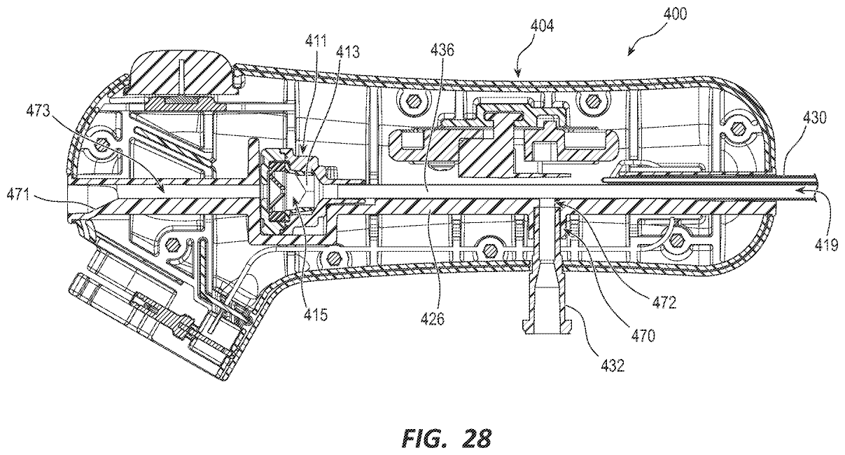

The housing 304 can define a pair of oppositely positioned openings 316 (one of which is visible in, e.g., FIG. 15) through which a steering wire actuator 318 can extend. The actuator 318 thus can extend outwardly from either side of the housing 304 to be accessible by a user. As shown in FIG. 17, each housing component 304a, 304b can define a recess 320 within which a portion of the actuator 318 can be received to permit rotation of the actuator 318 therein (see also FIG. 28).

With reference to FIGS. 16A-17, the distal end 308 of the housing 304 can include a distal aperture or opening 322, and the proximal end 306 of the housing 304 can include a proximal aperture or opening 324. The endoscope 300 can further include an adapter 326 positioned within the housing 304. As discussed further below, the adapter 326 can facilitate assembly of the endoscope 300 and/or can permit the housings of the endoscopes 300, 400 to be substantially identical to each other (e.g., with respect to shape, geometry, configuration, etc.), which can permit the use of the same molds in forming the housings and reduce manufacturing costs of the endoscopes 300, 400. A distal end of the adapter 326 can be coupled with the housing 304 and positioned within the distal opening 322, as shown in FIG. 16A, and a proximal end of the adapter 326 can be coupled with the housing 304 and positioned within the proximal opening 324, as shown in FIG. 16B.

With reference to FIGS. 16A and 17, the endoscope 300 can further include an insertion shaft 330, which can, in various embodiments, be of a construction the same as or similar to other insertion shafts discussed herein. In the illustrated embodiment, a proximal end of the insertion shaft 300 is coupled with the adapter 326. The insertion shaft 330 can extend distally from the adapter 326 and the housing 304.

With reference to FIGS. 15 and 17, the endoscope 300 can include a fluid port 331, which can be coupled with the housing 304 in any suitable manner. In the illustrated embodiment, the fluid port 331 includes a fluid connector 332, such as a luer fitting, that extends through an opening 334 defined by the housing 304. The fluid port 331 can be used to deliver fluid into a channeling tube 336 that is coupled with and extends through the insertion shaft 330, as discussed further below.

With reference to FIG. 16B, the endoscope 300 includes a mechanical connector 340 of any suitable variety. The mechanical connector 340 can be configured to permit selective attachment of the endoscope 300 to the reusable handle 500. In the illustrated embodiment, the mechanical connector 340 includes an opposing pair of latches 342 that are configured to deflect outwardly when advanced against a connector of the handle that includes outward protrusions (see FIG. 36) and snap back inwardly to seat the outward protrusions within recesses of the latches 342. Any other suitable selectively attachable and releasable mechanical connection interface is contemplated.

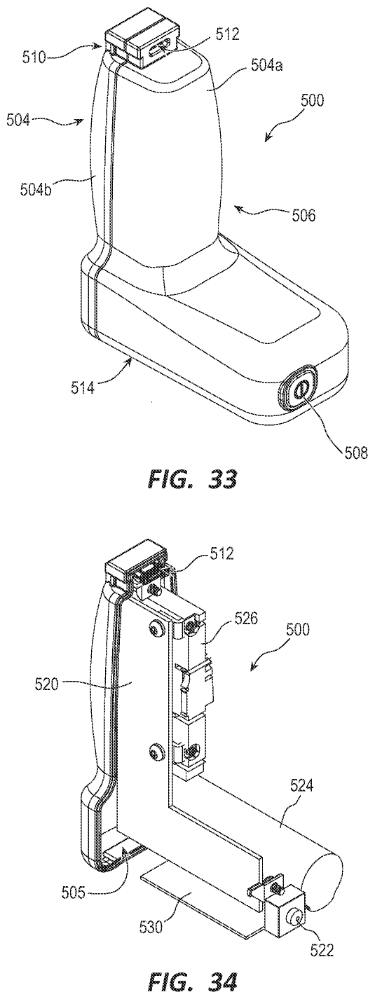

With continued reference to FIGS. 16B and 17, the endoscope 300 can include an electrical connector 346 of any suitable variety. The electrical connector 346 can electrically couple with an electrical connector of the handle 500 (see FIGS. 33-36). As discussed previously with respect to other insertion shafts, the insertion shaft 330 can include a camera 348 or other imaging sensor at a distal end thereof (see FIG. 23). One or more power and/or communication lines 350 can extend through the insertion shaft 330 and the housing 304 and be coupled with the connector 346. The one or more power and/or communication lines 350, which may be referred to as a power and/or communication line bundle, can include one or more electrical wires, such as may be used to provide power to the camera 348 and/or one or more LEDs or LED arrays, and/or may include one or more electrical, optical fiber, or other communication lines, such as may be used to communication information to and/or from the camera 348. The electrical connector 346 can be configured to permit the handle 500 to power the camera and, further, can permit communication between the camera and electrical componentry within the handle 500. Any suitable connector 346 is contemplated. In the illustrated embodiment, the electrical connector 346 comprises a USB-C interface.

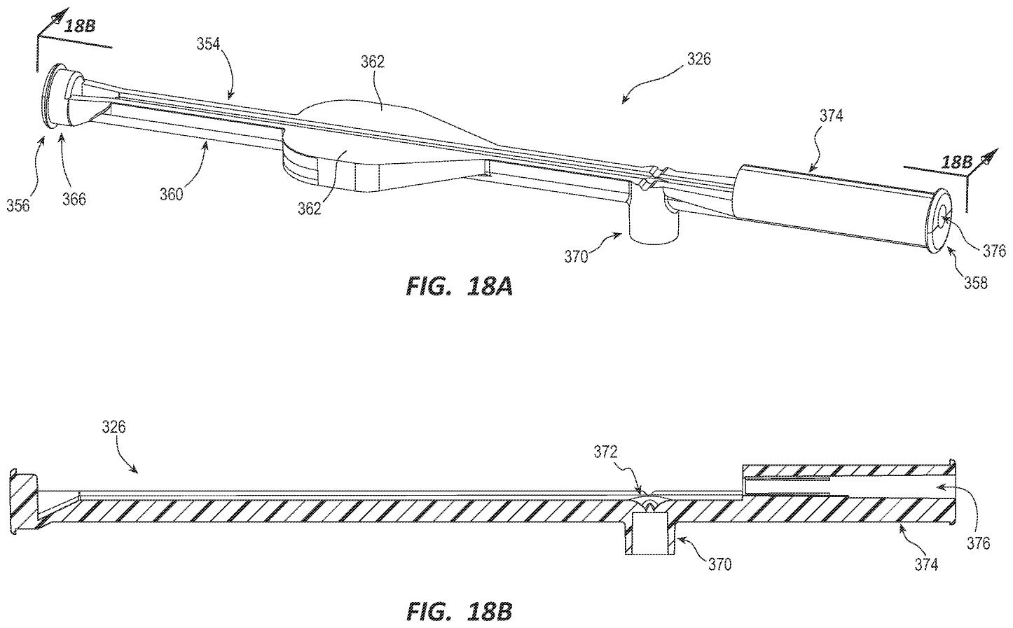

With reference to FIGS. 18A and 18B, the adapter 326 can include a longitudinally elongated body 354 that extends between a proximal tip 356 and a distal tip 358. The elongated body 354 can generally be relatively narrow along at least a majority of a full length thereof. In the illustrated embodiment, the body 354 includes an elongated shaft 360 and a pair of wings 362 that extend radially outwardly from the shaft 360.

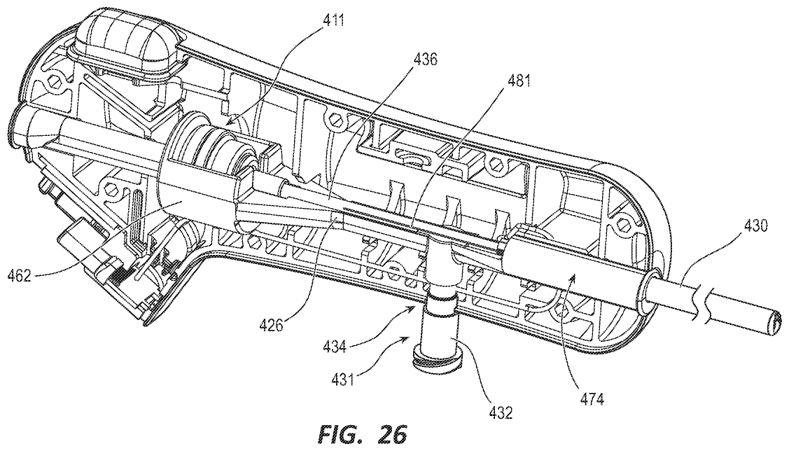

As shown in FIG. 17, the housing 340 can include a plurality of struts, ribs, supports, inward projections, or cross walls 367 that extend inwardly from the outer shell or sidewall of the housing elements 304a, 304b. Some cross walls 367 can define recesses 368. The wings 362 can extend into recesses 368 defined by the cross walls 367. As discussed further below with respect to FIGS. 26 and 28, the recesses 368 can be sized to accommodate one or more components (e.g., a valve) that are not present in the endoscope 300. The adapter 326 can situate internal components of the endoscope 300 within the housing 304, while an adapter 426 (discussed below) can situate internal components of the endoscope 400 within the housing thereof, which housing can be formed from the same molds used to make the housing 304.

As further discussed below, having a proximal opening in the housing can be useful for the endoscope 400, as this opening permits access to an instrument channel that extends through the housing and through the insertion shaft of the endoscope 400. The proximal opening 324 of the housing 304, however, is not needed, as no such instrument channel extends through the housing 304 or through the insertion shaft 330. If the proximal opening 324 were to be left open, a user might inadvertently attempt to insert an instrument through the opening and into the insertion shaft 330, but to no avail. In the illustrated embodiment, the adapter 324 includes a plug 366 at its proximal end, which fills the proximal opening 324 (see FIGS. 16B and 17) and thereby prevents access to the interior of the housing 304. The plug 366 can provide a visual cue and/or a physical barrier to warn against and/or prevent use of an elongated instrument with the endoscope 300, which use could instead be better suited for the endoscope 400.

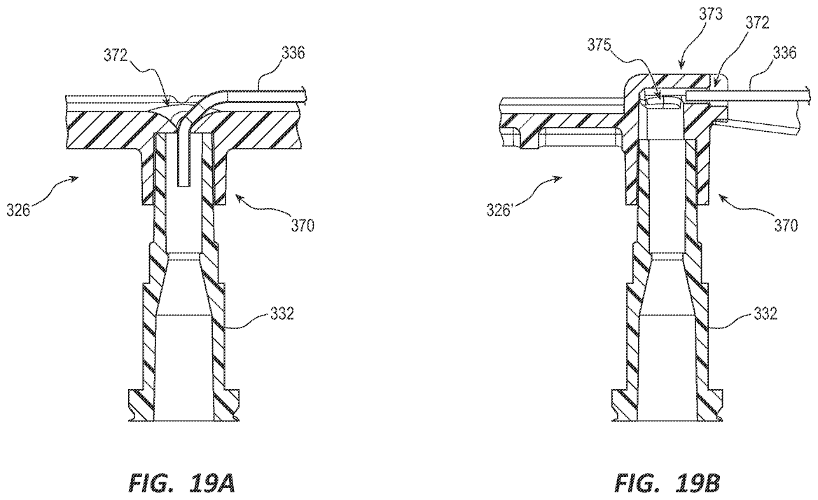

With reference to FIGS. 18A-19A, the adapter 326 can include a fluid socket, port, or aperture 370 that can be coupled with the fluid connector 332. A fluid-tight seal between the fluid connector 332 and the adapter 326 can be achieved in any suitable manner. In the illustrated embodiment, the adapter 326 further includes a narrowed neck 372 through which the channeling tube 336 passes. The neck 372 can be fluidically sealed about the outer surface of the channeling tube 336 in any suitable manner. In the illustrated embodiment, the channeling tube 336 extends through the neck 372 and into a lumen of the fluid connector 332, such that when a fluid delivery device is coupled to the fluid connector 332, such as for the delivery of saline, the fluid delivered from the fluid delivery device flows into the lumen of the fluid connector 332, into and through the open proximal end of the channeling tube 336 that resides within the lumen of the fluid connector 332, and distally through the channeling tube 336.

FIG. 19B depicts another embodiment of an adapter 326′ that includes a fluid socket, port, or aperture 370 that can be coupled with the fluid connector 332. A fluid-tight seal between the fluid connector 332 and the adapter 326 can be achieved in any suitable manner, such as, e.g., via adhesive. In the illustrated embodiment, the adapter 326 further includes boss 373 that defines a cavity 375. The boss 373 extends upward relative to the channeling tube 336 and defines the neck 372. The channeling tube 336 thus maintains a rectilinear profile in entering the narrowed neck 372 and into the cavity 375. Such an arrangement can avoid bending the channeling tube 336, which might result in kinking of the channeling tube 336. The neck 372 can be fluidically sealed about the outer surface of the channeling tube 336 in any suitable manner. Similar to the adapter 326 previously described, when a fluid delivery device is coupled to the fluid connector 332, such as for the delivery of saline, the fluid delivered from the fluid delivery device flows into the lumen of the fluid connector 332, through the cavity 375, into and through the open proximal end of the channeling tube 336 that resides within the lumen of the fluid connector 332, and distally through the channeling tube 336.

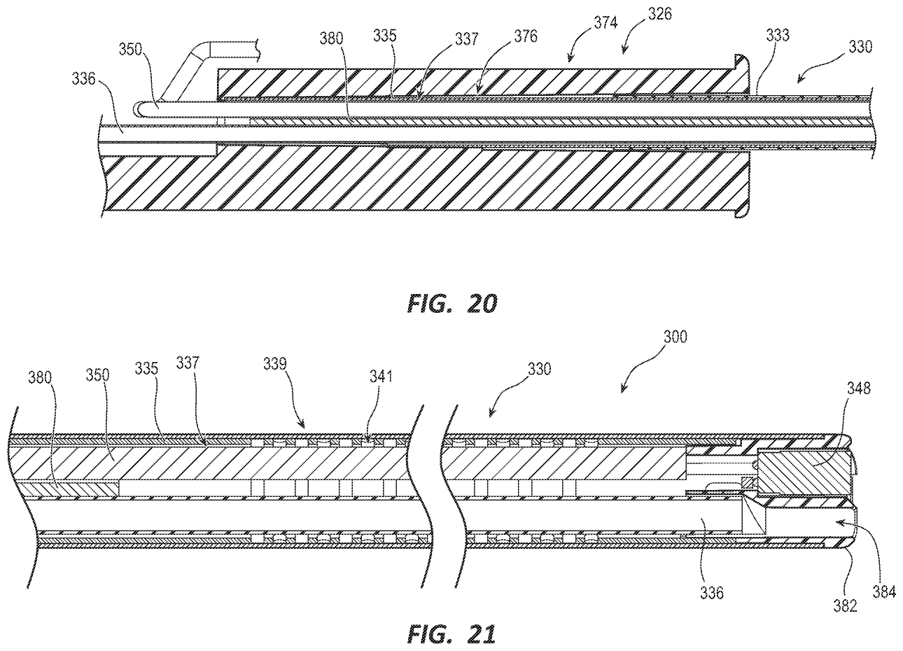

With reference to FIGS. 18B and 20, the adapter 326 can further include a barrel 374 at a distal end thereof. The barrel 374 can fit within and/or fill the distal opening 322 of the housing 304 (as shown in FIG. 17). The barrel 374 can define a longitudinally extending lumen 376, which can receive and connect with a proximal end of the insertion shaft 330. In the illustrated embodiment, the insertion shaft 330 includes a covering 333, which can include or resemble any of the coverings 104 previously discussed. The insertion shaft 330 can further include an insertion tube 335, which can include or resemble any of the tubes 103 previously discussed. For example, the insertion tube 335 can be relatively rigid and may be formed of metal. A distal portion of the insertion tube 335 may be laser cut to allow the insertion tube 335 to readily flex at a bending region 339 (FIG. 21) of the insertion shaft 330 in manners such as previously described.

In the illustrated embodiment, the insertion shaft 330 extends proximally by a greater amount than does the covering 333. Stated otherwise, a proximal end of the insertion shaft 330 is proximal to a proximal end of the covering 333. The proximal ends, including the proximal tips, of each of the insertion shaft 330 and the covering 333 are positioned within the lumen 376 and retained by the barrel 374 of the adapter 326.

The insertion tube 335 can define a lumen 337, which may also be referred to herein as a primary lumen. Each of the one or more power and/or communication lines 350, or stated otherwise, a power and/or communication line bundle or cable, can extend through the primary lumen 337. Similarly, the channeling tube 336 can also extend through the primary lumen 337.

As previously noted, fluid (e.g., saline) can be delivered into the channeling tube 336 via the fluid port 331 (see, e.g., FIG. 17), and can advance distally through the channeling tube 336 to a distal end of the insertion shaft 330 at which the channeling tube 336 terminates, as shown in FIG. 21. With further reference to FIG. 21 and additional reference to FIG. 23, the insertion tube 335 can be attached at a distal end thereof to a tip element 382. The tip element 382 can define a fluid port 384 that fluidly communicates with the channeling tube 336 and permits egress of fluid therefrom.

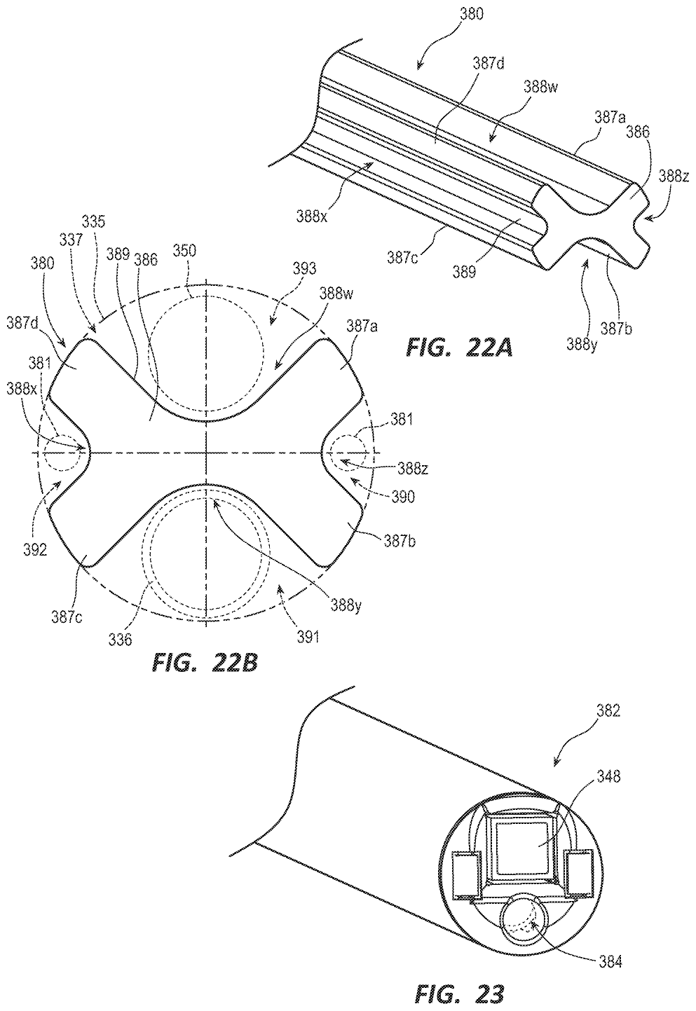

With reference to FIGS. 20 and 21, the endoscope 300 can include a guide member 380, which can manage positioning of one or more steering wires 381 (FIG. 22B), the power and/or communication lines 350, and/or the channeling tube 336. The guide member 380 can, in various embodiments, be the same as or similar to other embodiments of guide members 120 described above. For example, in some embodiments, the guide member 380 may be formed as a polymeric extrusion, which may have properties such as those previously described.

In the illustrated embodiment, a proximal tip of the guide member 380 is positioned within the insertion tube 335 and within the barrel 374 of the adapter 326. A distal tip of the guide member 380 is positioned within the insertion tube 335 at a position proximal to the bending section 339 of the insertion shaft 330. In FIG. 21, the bending section 330 is identifiable as the region in which the insertion tube 335 includes a plurality of laser cuts 341. In other embodiments, as previously discussed, the guide member 380 may extend through at least a portion of the bending section 339, and may, in further embodiments, be flexible in at least the bending section 339.

With reference to FIGS. 22A and 22B, the guide member 380 can include an elongated body 386. In some embodiments, a transverse cross-sectional profile of the body 386 may be constant along a full length of the body 386. For example, an end-on view of the body 386 may yield an outer profile that is the same as an outer profile that results from a transverse cross section at any position along the length of the body 386.

The body 386 can include an outer surface 389. In the illustrated embodiment, the outer surface 389 is continuous along a full periphery or perimeter of the body 386. In the illustrated embodiment, the body 386 is solid. Stated otherwise, the body 386 is devoid of any openings, channels, or passageways at an interior of the body 386, or within an interior of the outer surface 389. For example, in the view depicted in FIG. 22B, no openings or channels are present within the outer perimeter of the body 386 defined by the outer surface 389. Such may also be true of the body 386 along at least a majority of a length of the body 386.

The illustrated body 386 defines a substantially X-shaped perimeter. The X-shape is somewhat elongated in a horizontal direction in the illustrated orientation. The perimeter exhibits symmetry about two axes, which axes are shown in broken lines as vertical and horizontal axes, in the illustrated orientation.

The body 386 can include a plurality of longitudinally elongated arms or ribs 387a-387d. Concavely curving between each set of adjacent ribs 387a,b, 387b,c, 387c,d, and 387d,a is a respective longitudinally elongated recess or groove 388z, 388y, 388x, 388w. As shown in FIG. 22B, the guide member 380 can be positioned within the primary lumen 337 of the insertion tube 335 and may subdivide the primary lumen 337 into a plurality of smaller lumens, such as steering lumens 390, 392, a channeling lumen 391, and a power, data, electrical, communication, and/or auxiliary lumen 393. It may be said that each set of adjacent ribs 387a,b, 387b,c, 387c,d, and 387d,a cooperates with an internal surface of the insertion tube 335 to define the steering lumen 390, the channeling lumen 391, the steering lumen 392, and the auxiliary lumen 393, respectively. Alternatively, it may be said that each set of the recesses 388z, 388y, 388x, 388w cooperates with the internal surface of the insertion tube 335 to define the steering lumen 390, the channeling lumen 391, the steering lumen 392, and the auxiliary lumen 393, respectively. The lumens 390, 392 may be diametrically opposed, or at opposite sides of the guide member 380. The lumens 391, 393 may be diametrically opposed, or at opposite sides of the guide member 380.

In various embodiments, the body 386 cooperates with the insertion tube 335 to define 2, 3, 4, 5, or 6 lumens. In some embodiments, only steering lumens are formed to conduct steering wires therethrough. In other embodiments, steering lumens may not be present. For example, in some embodiments, only a channeling lumen and/or an auxiliary lumen are present.

In the illustrated embodiment, a central longitudinal axis of the insertion tube 335 extends through the body 386 of the guide member 380. The central longitudinal axis is the point of intersection of the vertical and horizontal lines, in the illustrated view.

In various embodiments, a transverse cross-sectional area of one or more of the lumens 391, 393, such as depicted in FIG. 22B, may be larger than a transverse cross-sectional area of one or more of the steering lumens 390, 392. In various embodiments, the transverse cross-sectional area of the channeling lumen 391 is (1) no less than about or equal to 2, 3, 4, 5, 6, or 7; (2) no more than about or equal to 2, 3, 4, 5, 6, or 7; (3) about or equal to 2, 3, 4, 5, 6, or 7; or (4) within a range of from about or equal to 2, 3, 4, 5, 6, or 7 to about or equal to 10 times the transverse cross-sectional area of one or more of the steering lumens 390, 392. In various embodiments, a maximum diametric height of the channeling lumen 391 is (1) no less than about or equal to 2, 3, 4, 5, 6, or 7; (2) no more than about or equal to 2, 3, 4, 5, 6, or 7; (3) about or equal to 2, 3, 4, 5, 6, or 7; or (4) within a range of from about or equal to 2, 3, 4, 5, 6, or 7 to about or equal to 10 times as large as a maximum diametric height of one or more of the steering lumens 390, 392, where diametric height is defined as a distance between the inner surface of the insertion tube 335 and the outer perimeter of the body of the guide member as measured along a diameter of the insertion tube 335. A larger channeling lumen may be present in embodiments of the endoscope 400, as further discussed below.

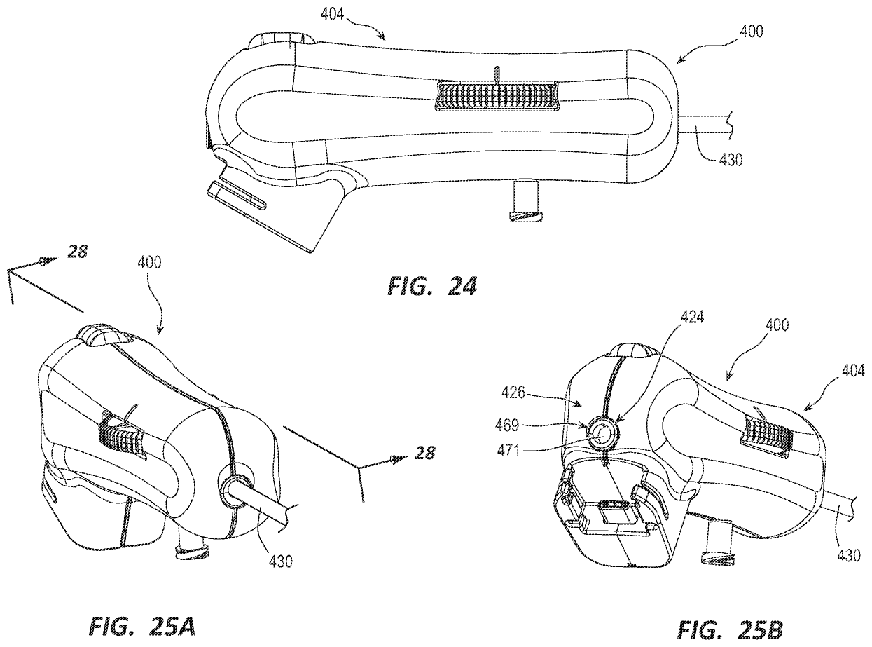

With reference to FIG. 24, the endoscope 400 can be substantially identical to the endoscope 300, but with modifications to allow for an instrument channel to pass through the endoscope 400. For example, in the views depicted in FIGS. 15 and 24, the endoscopes 300, 400 appear to be substantially identical, except that an insertion shaft 430 of the endoscope 400 has a greater outer diameter than does the insertion shaft 330 of the endoscope 330. Similarly, in the views depicted in FIGS. 16A and 25A, the endoscopes 300, 400 appear to be substantially identical, except for the larger size of the insertion shaft 430. Although not identified with numbering in FIG. 25B, the endoscope 400 can, in some embodiments, include mechanical and electrical connectors that are identical to those of the endoscope 300.

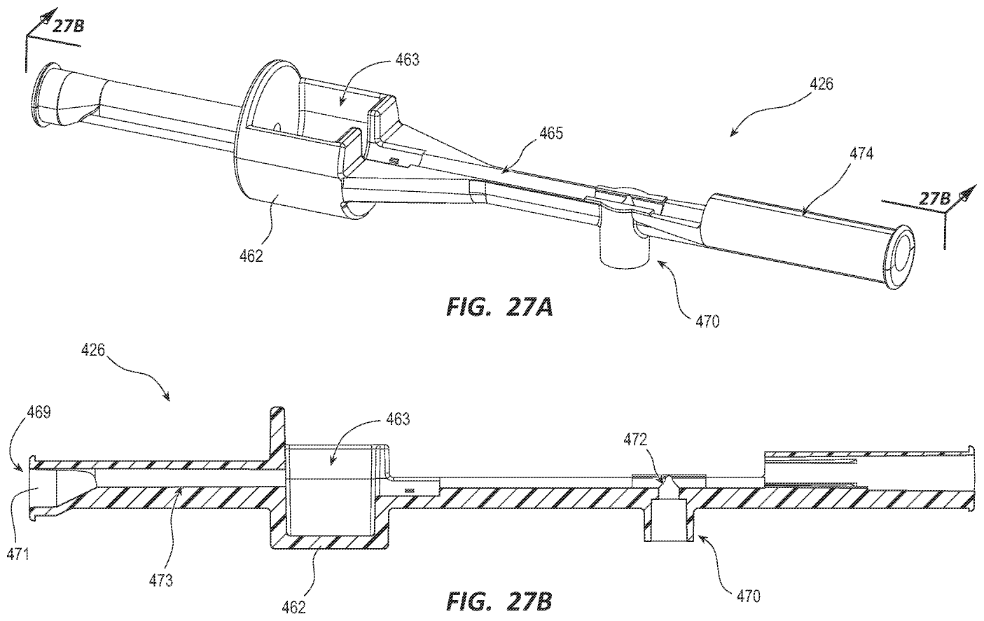

With reference to FIG. 25B, a proximal opening 424 in the housing 404 is shown. A proximal end of an adapter 426 that is also visible varies from a proximal end of the adapter 326. In particular, the adapter 426 includes an instrument port 469 configured to receive one or more instruments into an instrument channel through the endoscope, as further discussed below. As shown in FIGS. 25B and 27B, the instrument port 469 can include an entryway 471 that can assist in channeling the instrument distally into the instrument channel. In the illustrated embodiment, the entryway 471 is substantially funnel shaped.

With reference to FIGS. 26-29, the adapter 426 can further include a receptacle 462 that is configured to receive therein at least a portion of a valve assembly 411. In particular, the receptacle 462 defines a cavity 463 sized to receive the valve assembly 411. Cross walls of the housing 404, such as previously described with respect to the housing 304, include recesses to accommodate the receptacle 462.

With reference to FIGS. 27A and 27B, the adapter 426 can define the instrument port 469 and entryway 471, which can lead to a proximal lumen 473. The proximal lumen 473 terminates at the cavity 463 of the receptacle 462. Distal to the receptacle 462 is a groove 465, which can be sized to receive a channeling tube 436 (see FIGS. 26 and 28). The channeling tube 436 can lead distally to a distal barrel 474, which can resemble the barrel 374. The barrel 474 and an internal lumen defined thereby (such as the lumen 376) of the adapter 436 can be larger than the corresponding features of the adapter 326. These features may be enlarged to accommodate a relatively larger channeling tube 436, as the channeling tube 436 defines a lumen sufficiently large to accommodate any of a variety of suitable elongated instruments. In various embodiments, the channeling tube 436 may be sized to accommodate instruments sized up to 4, 5, 6, or 7 French.

With reference to FIGS. 26 and 28, more generally speaking, an instrument channel 419 that extends through a full length of the endoscope 400 can be sized to accommodate any of a variety of suitable elongated instruments. In various embodiments, the instrument channel 419 may be sized to accommodate instruments sized up to 4, 5, 6, or 7 French.

The instrument channel 419 may collectively be defined by the instrument port 469 and entryway 471 of the adapter 426, the valve assembly 411 (including an internal chamber 415 thereof, which includes therein any suitable valve member 413), the channeling tube 436 (which is coupled to the valve assembly 411), and a distal port through a tip 431 of the endoscope 400, as shown in FIGS. 30 and 32. All of the instrument-channel defining components within the housing 404 may be aligned such that the instrument channel 419 extends rectilinearly through the housing 404.

With reference to FIGS. 26-28, the endoscope 400 can include a fluid port 431 such as the fluid port 331 described above. The fluid port 431 can extend through an opening 434 through the housing, and can include a connector 432. As shown in FIG. 28A, in some embodiments, the adapter 426 includes an aperture 470 and a neck 472 that coupled to the connector 432. In some instances, an opening into a sidewall of the channeling tube 436 may be provided by passing a drill bit or other suitable instrument through the connector 432 and the neck 470. In some embodiments, the channeling tube 436 may be fluidically sealed to the adapter 426 in at least the region of the neck 470, such as, e.g., via adhesive.

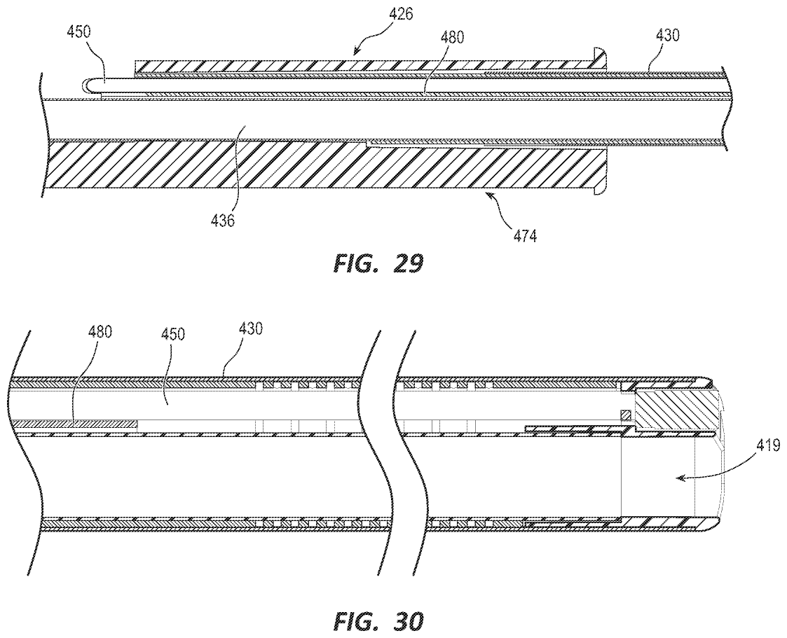

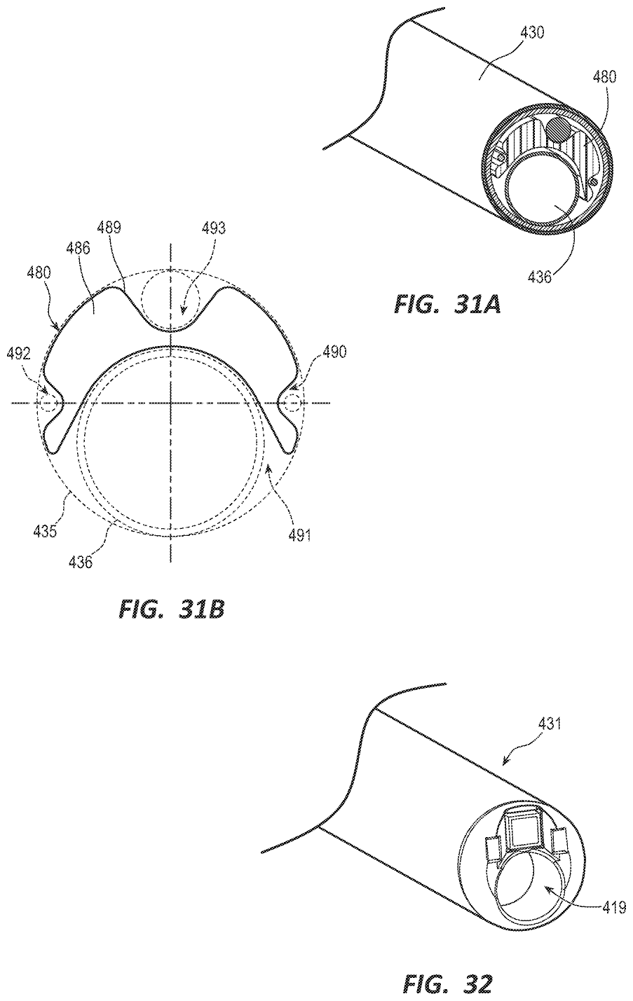

FIGS. 29 and 30 depict an arrangement similar to that depicted in FIGS. 20 and 21. A guide element 480 is shown, which extends through the barrel 474 of the adapter 426 at a proximal end thereof. A distal end of the guide element 480 terminates at a position proximal to a bending region of the insertion shaft 430. The channeling tube 436 extends through the insertion shaft 430 adjacent to the guide element 480. One or more power and/or communication lines 480 may similarly extend through the insertion shaft 430 adjacent to the guide element 480. As shown in FIG. 31A, two diametrically opposed steering wires (not identified with numbering) similarly extend through the insertion shaft 430 adjacent to the guide element 480.

The guide element 480 can include an elongated body 486 that includes a plurality of elongated ribs and grooves, such as previously discussed. The shapes and sizes of the ribs and grooves, however, are different. For example, the ribs and grooves may be shaped to accommodate a relatively larger channeling tube 436, while maintaining two steering wires in diametric opposition. In other or further instances, the ribs and grooves may be shaped to accommodate the relatively larger channeling tube 436 while maintaining contact with the inner surface of the tube 436 along at least a portion of each of four quadrants of the insertion tube 435, which may provide, e.g., reinforcement to the tube 435.

An outer surface 489 of the body 486 may define a transverse cross-sectional perimeter that is symmetrical along one axis but asymmetrical along an orthogonal axis. Other amounts of asymmetry are contemplated.

As with other embodiments discussed herein, the ribs and/or grooves of the body 486 can cooperate with the inner surface of the tube 435 to define a plurality of lumens 490, 491, 492, 493. Other and further illustrative descriptions of arrangements such as that depicted in FIG. 31B are included in certain of the examples below, including descriptions of the body 486 and/or the lumens 490, 491, 492, 493 at least partially defined thereby.

With reference to FIGS. 33 and 34, in certain embodiments, the handle 500 can include a housing 504. The housing 504 can be configured to be releasably connect with each of the endoscopes 300, 400, as previously discussed. The housing 504 can include a pair of housing elements or components 504a, 504b that can cooperate to define an interior space 505. The components 504a, 504b may be joined in a fluid-tight manner to prevent ingress of cleaning fluid into the handle 500 during cleaning by submersion in a cleaning solution. In the illustrated embodiment, a power button cover 508 may be overmolded to the housing element 504a.

The handle 500 may be capable of resting upright on a base surface 514. The handle 500 can include a gripping region 506 defined by the housing 504. The gripping region 506 can be generally convex and may fit ergonomically within the hand of a user.

The housing 504 can define a mechanical connector 510 at an upper end thereof, which may be configured to couple with the mechanical connectors of the endoscopes 300, 400. For example, as shown in FIG. 36, the mechanical connector 510 may include a planar surface 555. This planar surface 555 can correspond to a coupling plane P_{C}, shown in FIG. 15, along which the handle 500 and either endoscope 300, 400 can be moved relative to each other for coupling or decoupling. With continued reference to FIG. 15, the coupling plane P_{C} may be substantially orthogonal to an axis of elongation of the handle A_{H}. Also shown in FIG. 15 is the relative geometry of the axis of elongation of the handle A_{H} and the central longitudinal axis of the endoscope 300. These axes may, in some embodiments, be at an oblique angle relative to each other.

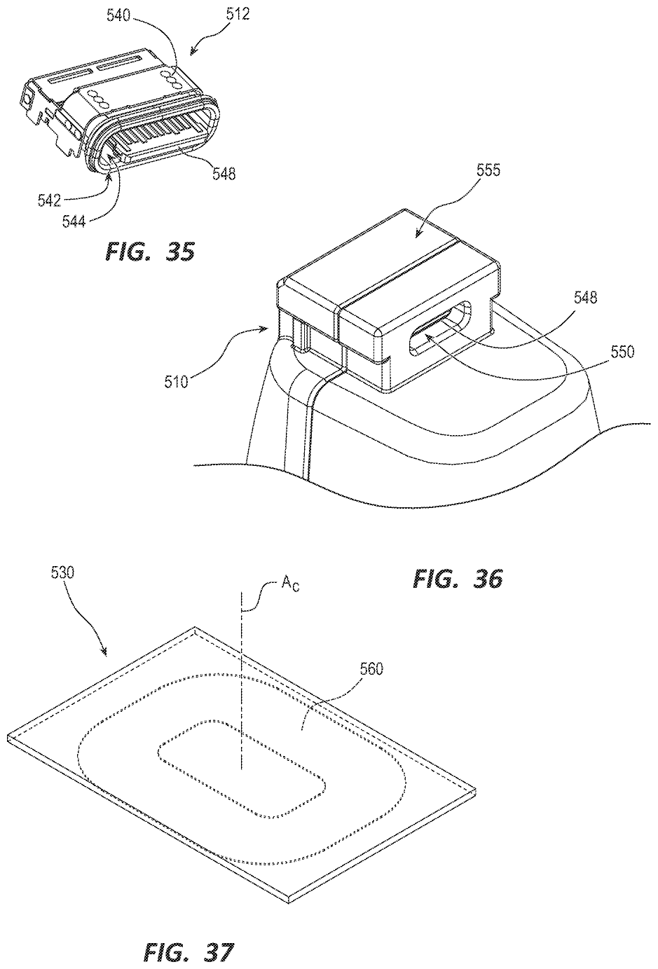

With reference again to FIG. 33, the handle 500 can further include an electrical connector 512 of any suitable variety, such as previously disclosed. For example, as shown in FIGS. 34 and 35, the electrical connector 512 may be a USB-C connector 540. The connector 540 may be coupled with the housing 504 via any suitable fluid-tight seal. For example, in various embodiments, an O-ring, potting adhesive, and/or other sealing elements may be used. The connector 540 can define an opening 542 and a recess 544 that extends inwardly from the opening 542. For example, the recess 544 may extend proximally away from a distal edge 548 of the connector 512. Stated otherwise, the recess 544 may extend inwardly toward the interior space of the housing 504. As shown in FIG. 36, the housing may define an opening 550 that aligns with the opening 542 of the connector 512.

With reference to FIG. 34, the handle can include within the interior space 505 a variety of components, such as a rechargeable battery 524, a weight 526 that can assist in maintaining the handle submerged in a cleaning solution, and any other suitable circuitry 520, which may include a printed circuit board and/or appropriate wireless transceiver componentry. The illustrated embodiment further includes an inductive coil 530 that is configured to inductively recharge the battery 524.

As shown in FIG. 34, the inductive coil 530 may have a substantially planar arrangement, and may be positioned flatly adjacent to the base 514 of the housing. As shown in FIG. 37, the induction coil may include a plurality of induction elements 560 that may be said to coil along coiling plane. In the illustrated embodiment, the coiling plane is substantially parallel to a surface on which the base 514 rests when the handle 500 is set upright. The induction elements 560 can be coiled around a coiling axis Ac that is normal to the coiling plane.

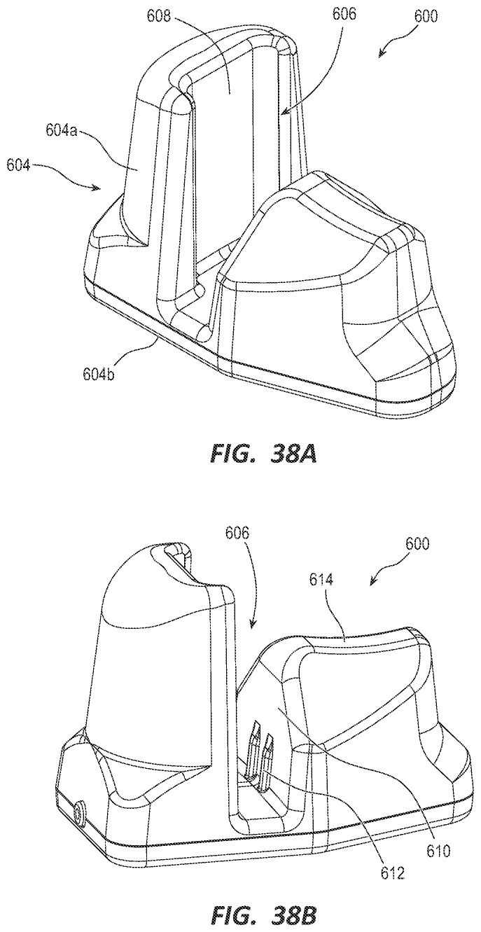

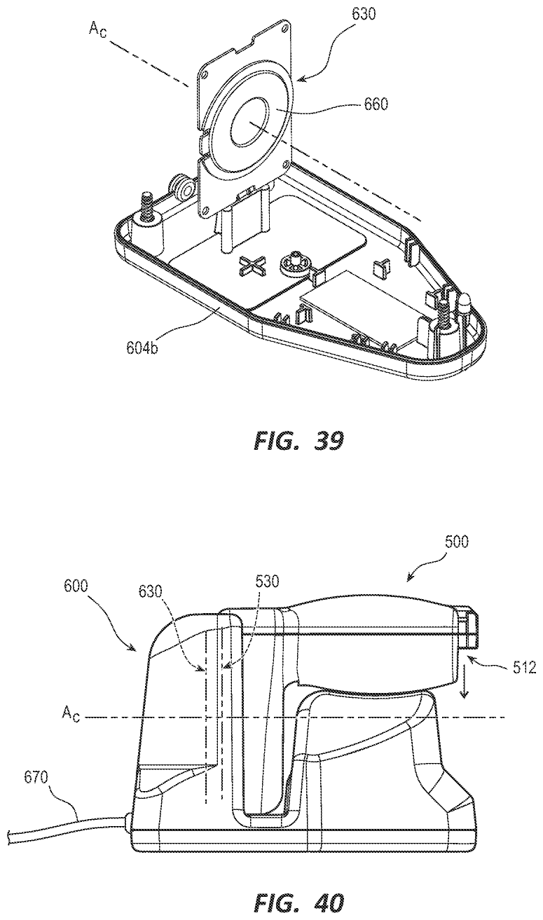

With reference to FIGS. 38A-40, the charging cradle 600 can include a housing 600 that comprises an upper housing component 604a and a base 604b. The housing 600 defines an external receptacle 606 configured to receive the handle 500. In particular, in the illustrated embodiment, the receptacle 606 is configured to receive at least a portion of the base 514 of the handle 500.

The charging cradle 600 includes an inductive coil 630 positioned within the housing 604. The inductive coil 630 can include a plurality of coil elements that coil around a coiling axis Ac that is normal to the coiling plane.

With reference to FIG. 40, when the charging cradle 600 rests on the base of its housing 604 and the handle 500 is received in the receptacle of the charging cradle housing 604, the inductive coils 530, 630 of the handle 500 and the charging cradle 600 are oriented to permit inductive charging of the rechargeable battery and the opening of the electrical connector is oriented lower than the recess of the electrical connector to permit cleaning fluid to egress from the electrical connector under the influence of gravity.

Other or further arrangements of illustrative embodiments of the handle 500 and the charging cradle 600 are provided in Examples 101 to 107 below, which may be understood in the context of, e.g., FIGS. 33-40.

As also shown in FIG. 40, the charging cradle 600 can include a power cord 670 of any suitable variety to power the inductive coil 630.

### Examples

The present paragraph recites 107 illustrative examples of endoscopes and systems that correspond with and/or may provide further detail with respect to various embodiments of the foregoing written description and/or the illustrative drawings. In these examples, the terminology “Example X to Example Y” means Example X through Example Y, and thus includes the endpoints of the recited range of examples. Moreover, embodiments capable of derivation from the various Examples that follow are also expressly incorporated into the present written description. These additional embodiments are determined by replacing the dependency of a given dependent Example with the phrase “any one of Example [x] through the preceding Example,” where the bracketed term “[x]” is replaced with the number of the most recently recited independent Example. For example, for the first Example set that begins with independent Example 1, Example 3 can depend from either of Examples 1 or 2, with these separate dependencies yielding two distinct embodiments; Example 4 can depend from any one of Examples 1, 2, or 3, with these separate dependencies yielding three distinct embodiments; Example 5 can depend from any one of Examples 1, 2, 3, or 4, with these separate Examples yielding four distinct embodiments; and so on.
Example 1. An endoscope comprising:
   a handle;
   an actuator coupled to the handle;
   an elongated shaft coupled to the handle, the shaft comprising:
      a deflectable distal portion;
      an imaging assembly at a distal end of the shaft;
      a first tube that defines a hollow interior; and
      an elongated guide member positioned within the interior of the first tube, the guide member comprising:
         a solid interior such that a transverse cross section of the guide member along at least a portion of a full longitudinal length of the guide member is devoid of any internal openings;
         a first longitudinal groove at an external surface of the guide member that cooperates with the first tube to define a first lumen; and
         a second longitudinal groove at the external surface of the guide member that cooperates with the first tube to define a second lumen through which communication lines associated with the imaging assembly pass; and
   a steering wire coupled to the deflectable distal portion of the shaft, extending through the first lumen, and coupled with the actuator such that actuation of the actuator causes the steering wire to deflect the distal portion of the shaft.
Example 2. The endoscope of Example 1, wherein the guide member comprises a third longitudinal groove at the external surface of the guide member that cooperates with the first tube to define a third lumen through which a second tube passes.
Example 3. The endoscope of Example 2, wherein the handle comprises a fluid port coupled with the second tube, the fluid port being configured to deliver fluid into the second tube for advancement of the fluid toward the distal end of the shaft through the third lumen.
Example 4. The endoscope of Example 2 or Example 3, wherein the handle comprises an instrument port coupled with the second tube, the instrument port being configured to receive an elongated instrument therethrough and into the second tube for advancement of the instrument toward the distal end of the shaft through the third lumen.
Example 5. The endoscope of any one of Example 2 to Example 4, wherein a central longitudinal axis of the first tube extends through the second tube.
Example 6. The endoscope of any one of Example 2 to Example 5, wherein a central longitudinal axis of the first tube extends through the third lumen.
Example 7. The endoscope of any one of Example 2 to Example 6, wherein an inner diameter of the second tube is greater than an inner radius of the first tube.
Example 8. The endoscope of any one of Example 2 to Example 7, wherein a central longitudinal axis defined by the first tube runs parallel to a central longitudinal axis defined by the second tube.
Example 9. The endoscope of any one of Example 2 to Example 8, wherein the guide member further comprises a fourth longitudinal groove at the external surface of the guide member that cooperates with the first tube to define a fourth lumen through which an additional steering wire extends.
Example 10. The endoscope of Example 9, wherein the first and fourth lumens are diametrically opposed.
Example 11. The endoscope of Example 9 or Example 10, wherein the second and third lumens are diametrically opposed.
Example 12. The endoscope of any one of Example 9 to Example 11, wherein at least a portion of the third lumen is positioned on a line that extends through the first and fourth lumens.
Example 13. The endoscope of Example 1, wherein the guide member comprises an extrusion of a polymeric material.
Example 14. The endoscope of Example 1, wherein the transverse cross section of the guide member is substantially X-shaped.
Example 15. The endoscope of Example 1 or Example 14, wherein a central longitudinal axis of the first tube extends through the guide member.
Example 16. The endoscope of any one of Example 1 to Example 15, wherein the first tube comprises a rigid portion that extends proximally from the deflectable distal portion of the shaft.
Example 17. The endoscope of Example 16, wherein the first tube comprises a bending section that corresponds with at least a portion of the deflectable distal portion of the shaft.
Example 18. The endoscope of Example 17, wherein the bending section of the first tube comprises a plurality of laser-cut grooves.
Example 19. The endoscope of Example 17, wherein the guide member comprises a distal tip that terminates at a position at or proximal to a proximal end of the bending section.
Example 20. The endoscope of Example 17, wherein the guide member extends through at least a portion of each of the rigid portion and the bending section.
Example 21. The endoscope of any one of Example 1 to Example 20, wherein the handle is separable into a reusable portion and a disposable portion.
Example 22. The endoscope of Example 21, wherein the reusable portion comprises circuitry for wireless communication with a processor and/or a display monitor.
Example 23. The endoscope of Example 21 or Example 22, wherein the disposable portion comprises the actuator.
Example 24. The endoscope of any one of Example 1 to Example 23, wherein the handle defines a channel through which elongated instruments may be passed.
Example 25. The endoscope of Example 24, wherein the endoscope further comprises a working channel, and wherein the channel defined by the handle constitutes a proximal end of the working channel.
Example 26. The endoscope of Example 25, wherein a portion of the working channel is defined by a separate accessory that is selectively attachable to and detachable from the handle.
Example 27. The endoscope of Example 26, wherein the accessory includes a rigid distal end configured to resist deformation.
Example 28. The endoscope of Example 26, wherein the accessory includes a bending section that is more bendable than at least a portion of the accessory that is immediately proximal to the bending section.
Example 29. The endoscope of any one of Example 1 to Example 28, wherein the shaft comprises a proximal portion that extends from the handle to a bending region that permits deflection of the distal portion, wherein the proximal portion is rigid.
Example 30. The endoscope of any one of Example 1 to Example 28, wherein the shaft comprises a proximal portion that extends from the handle to a bending region that permits deflection of the distal portion, wherein the proximal portion is flexible.
Example 31. An endoscope comprising:
a tube comprising an inner surface that defines a primary lumen;
first and second steering wires positioned within the primary lumen of the tube; and
a guide member positioned within the primary lumen of the tube, the guide member comprising:
   an elongated body that defines an outer perimeter, the body being solid such that an entirety of a region internal to the outer perimeter is devoid of any passageways;
   a plurality of ribs extending longitudinally along the body to define at least a portion of the outer perimeter of the body;
   a first steering lumen positioned angularly between a first pair of the plurality of ribs, the first steering lumen being defined by a first portion of the outer perimeter of the elongated body and a first portion of the inner surface of the tube, the first steering wire extending longitudinally through the first steering lumen; and
   a second steering lumen positioned angularly between a second pair of the plurality of ribs, the second steering lumen being defined by a second portion of the outer perimeter of the elongated body and a second portion of the inner surface of the tube, the second steering wire extending longitudinally through the second steering lumen.
Example 32. The endoscope of Example 31, wherein the first and second steering lumens are diametrically opposed.
Example 33. The endoscope of Example 31, further comprising a channeling lumen positioned angularly between a third pair of the plurality of ribs, the channeling lumen being defined by a third portion of the outer perimeter of the elongated body and a third portion of the inner surface of the tube.
Example 34. The endoscope of Example 33, wherein a single rib of the plurality of ribs is one of the ribs that constitutes the first pair of ribs and is also one of the ribs that constitutes the third pair of ribs.
Example 35. The endoscope of Example 33, wherein the channeling lumen is angularly adjacent to each of the first and second steering lumens.
Example 36. The endoscope of Example 33, wherein the channeling lumen defines a transverse cross-sectional area that is at least three times greater than a transverse cross-sectional area of each of the first and second steering lumens.
Example 37. The endoscope of Example 33, wherein a maximum diametric height of the channeling lumen is at least three times as large as a maximum diametric height of one or more of the first and second steering lumens, wherein diametric height is defined as a distance between the inner surface of the tube and the outer perimeter of the body of the guide member as measured along a diameter of the tube.
Example 38. The endoscope of Example 33, further comprising a channeling tube, wherein the channeling tube extends longitudinally through the channeling lumen.
Example 39. The endoscope of Example 38, wherein the channeling tube is configured to accommodate passage therethrough of an instrument of up to 7 French.
Example 40. The endoscope of Example 38, wherein the channeling tube is fluidly connected to a fluid port and is configured to permit passage therethrough of fluid delivered into the channeling tube via the fluid port.
Example 41. The endoscope of Example 33, wherein a portion of the channeling lumen is positioned between a portion of each of the first and second steering lumens along a diameter of the tube.
Example 42. The endoscope of Example 33, wherein a portion of the channeling lumen is positioned linearly between an entirety of the first and second steering lumens.
Example 43. The endoscope of Example 33, further comprising an electrical lumen positioned angularly between a fourth pair of the plurality of ribs, the electrical lumen being defined by a fourth portion of the outer perimeter of the elongated body and a fourth portion of the inner surface of the tube, wherein one or more electrical lines extend through the electrical lumen.
Example 44. The endoscope of Example 43, wherein the electrical lumen is angularly adjacent to each of the first and second steering lumens.
Example 45. The endoscope of Example 43, wherein the electrical lumen and the channeling lumen are at opposite sides of the guide member along a diameter of the tube.
Example 46. The endoscope of Example 31, wherein a transverse cross-sectional shape of the guide member comprises at least two axes of symmetry.
Example 47. The endoscope of Example 31, wherein a transverse cross-sectional shape of the guide member has only one single axis of symmetry.
Example 48. The endoscope of Example 31, wherein an outermost surface of each of the plurality of ribs contacts the inner surface of the tube along at least a majority of a longitudinal length of the guide member.
Example 49. The endoscope of Example 31, wherein the guide member interacts with a sidewall of the tube to reinforce the tube to inhibit bending of the tube.
Example 50. The endoscope of Example 31, wherein the tube comprises a bending section, and wherein a distal tip of the guide member is positioned proximal to the bending section.
Example 51. The endoscope of Example 31, wherein the tube comprises a bending section, and wherein a portion of the guide member extends through at least a portion of the bending section.
Example 52. An endoscope comprising:
a first tube comprising an inner surface that defines a primary lumen;
a channeling tube positioned within the primary lumen of the tube;
first and second steering wires positioned within the primary lumen of the tube; and
a guide member positioned within the primary lumen of the tube, the guide member comprising:
   an elongated body that defines an outer perimeter;
   a plurality of ribs extending longitudinally along the body to define at least a portion of the outer perimeter of the body;
   a first steering lumen positioned angularly between a first pair of the plurality of ribs, the first steering lumen being defined by a first portion of the outer perimeter of the elongated body and a first portion of the inner surface of the tube, the first steering wire extending longitudinally through the first steering lumen;
   a second steering lumen positioned angularly between a second pair of the plurality of ribs, the second steering lumen being defined by a second portion of the outer perimeter of the elongated body and a second portion of the inner surface of the tube, the second steering wire extending longitudinally through the second steering lumen; and
   a channeling lumen positioned angularly between a third pair of the plurality of ribs, the channeling lumen being defined by a third portion of the outer perimeter of the elongated body and a third portion of the inner surface of the tube, the channeling tube extending longitudinally through the channeling lumen.
Example 53. The endoscope of Example 52, wherein the first and second steering lumens are diametrically opposed.
Example 54. The endoscope of Example 52, wherein a single rib of the plurality of ribs is one of the ribs that constitutes the first pair of ribs and is also one of the ribs that constitutes the third pair of ribs.
Example 55. The endoscope of Example 52, wherein the channeling lumen is angularly adjacent to each of the first and second steering lumens.
Example 56. The endoscope of Example 52, wherein the channeling lumen defines a transverse cross-sectional area that is at least three times greater than a transverse cross-sectional area of each of the first and second steering lumens.
Example 57. The endoscope of Example 52, wherein a maximum diametric height of the channeling lumen is at least three times as large as a maximum diametric height of one or more of the first and second steering lumens, wherein diametric height is defined as a distance between the inner surface of the first tube and the outer perimeter of the body of the guide member as measured along a diameter of the first tube.
Example 58. The endoscope of Example 52, further comprising a channeling tube, wherein the channeling tube extends longitudinally through the channeling lumen.
Example 59. The endoscope of Example 58, wherein the channeling tube is configured to accommodate passage therethrough of an instrument of up to 7 French.
Example 60. The endoscope of Example 58, wherein the channeling tube is fluidly connected to a fluid port and is configured to permit passage therethrough of fluid delivered into the channeling tube via the fluid port.
Example 61. The endoscope of Example 52, wherein a portion of the channeling lumen is positioned between a portion of each of the first and second steering lumens along a diameter of the first tube.
Example 62. The endoscope of Example 52, wherein a portion of the channeling lumen is positioned linearly between an entirety of the first and second steering lumens.
Example 63. The endoscope of Example 52, further comprising an electrical lumen positioned angularly between a fourth pair of the plurality of ribs, the electrical lumen being defined by a fourth portion of the outer perimeter of the elongated body and a fourth portion of the inner surface of the first tube, wherein one or more electrical lines extend through the electrical lumen.
Example 64. The endoscope of Example 63, wherein the electrical lumen is angularly adjacent to each of the first and second steering lumens.
Example 65. The endoscope of Example 63, wherein the electrical lumen and the channeling lumen are at opposite sides of the guide member along a diameter of the first tube.
Example 66. The endoscope of Example 52, wherein a transverse cross-sectional shape of the guide member comprises at least two axes of symmetry.
Example 67. The endoscope of Example 52, wherein a transverse cross-sectional shape of the guide member has only one single axis of symmetry.
Example 68. The endoscope of Example 52, wherein an outermost surface of each of the plurality of ribs contacts the inner surface of the tube along at least a majority of a longitudinal length of the guide member.
Example 69. The endoscope of Example 52, wherein the guide member interacts with a sidewall of the tube to reinforce the tube to inhibit bending of the tube.
Example 70. The endoscope of Example 52, wherein the tube comprises a bending section, and wherein a distal tip of the guide member is positioned proximal to the bending section.
Example 71. The endoscope of Example 52, wherein the tube comprises a bending section, and wherein a portion of the guide member extends through at least a portion of the bending section.
Example 72. A system comprising:
a handle configured to be held by a hand of a user, the handle comprising:
   an electrical connector;
   electrical circuitry in electrical communication with the electrical connector;
   a mechanical connector; and
a first endoscope and a second endoscope, each of the first and second endoscopes, respectively, defining a longitudinal axis and comprising:
   an electrical connector configured to releasably couple with the electrical connector of the handle;
   a mechanical connector configured to releasably couple with the mechanical connector of the handle;
   a steerable insertion shaft elongated along the longitudinal axis, the insertion shaft comprising a camera at a distal end thereof that is electrically coupled with the electrical connector of the endoscope;
   a housing elongated along the longitudinal axis between a proximal end and a distal end, the housing being coupled with the insertion shaft such that the insertion shaft extends distally from the housing;
   an actuator coupled with the housing and with steering wires that extend through the insertion shaft such that physical manipulation of the actuator causes deflection of a distal portion of the insertion shaft,
wherein the first endoscope comprises an instrument channel that extends through the proximal and distal ends of the housing of the first endoscope and through the insertion shaft of the first endoscope, the instrument channel being configured to accommodate passage therethrough of an elongated instrument that is configured to extend beyond a distal tip of the insertion shaft of the first endoscope,
wherein the second endoscope is devoid of any instrument channel that extends through the proximal and distal ends of the housing of the second endoscope, and
wherein each of the first and second endoscopes is configured to individually couple with the handle via the electrical and mechanical connectors of the respective endoscope.
Example 73. The system of Example 72, wherein each of the first and second endoscopes is disposable.
Example 74. The system of Example 73, wherein the handle is reusable.
Example 75. The system of Example 72, wherein the handle is reusable.
Example 76. The system of Example 75, wherein the handle comprises a housing and electronic components at an interior of the housing, and wherein the housing is fluidically sealed such that the handle is fully submersible in a cleaning fluid without any portion of the cleaning fluid entering into the interior of the housing.
Example 77. The system of Example 76, wherein the electrical connector of the handle comprises a USB-C connector.
Example 78. The system of Example 72, wherein the first endoscope further comprises a valve assembly that defines a portion of the instrument channel.
Example 79. The system of Example 78, wherein the first endoscope further comprises a fluid port in fluid communication with the instrument channel at a position distal to the valve assembly, wherein the fluid port is configured to deliver fluid into the instrument channel and the valve assembly is configured to prevent fluid from traveling proximally beyond the valve assembly to promote advancement of the fluid toward the distal end of the insertion shaft through the instrument channel.
Example 80. The system of Example 72, wherein the first endoscope further comprises a port at the proximal end of the housing that defines a proximal end of the instrument channel.
Example 81. The system of Example 80, wherein the port comprises a funnel-shaped entryway that narrows in a distal direction to assist in directing the elongated instrument into the instrument channel.
Example 82. The system of Example 72, wherein the housing of the second endoscope comprises a closed proximal end to prevent insertion of any instruments therethrough.
Example 83. The system of Example 72, wherein the second endoscope further comprises a fluid port in fluid communication with a fluid delivery tube that extends through the insertion shaft.
Example 84. The system of Example 72, wherein the electrical circuitry comprises one or more of a rechargeable battery and a wireless transceiver.
Example 85. The system of Example 72, wherein the handle is elongated along an additional longitudinal axis that extends at an oblique angle relative to the longitudinal axis of the first endoscope when the handle is coupled with the first endoscope or relative to the longitudinal axis of the second endoscope when the handle is coupled with the second endoscope, respectively.
Example 86. The system of Example 72, wherein the handle is elongated along an additional longitudinal axis, and wherein coupling of the handle with either the first or second endoscope is achieved by effecting relative movement of the handle and the first or second endoscope along a plane that is orthogonal to said additional longitudinal axis.
Example 87. The system of Example 72, wherein:
the housing of the first endoscope defines an internal cavity through which a portion of the steering wires of the first endoscope extend and through which a portion of the instrument channel extends; and
the housing of the second endoscope defines an internal cavity through which a portion of the steering wires of the second endoscope extend,
wherein the housings of the first and second endoscopes are substantially identical to each other.
Example 88. The system of Example 87, wherein the first endoscope further includes an adapter within the housing of the first endoscope that extends between the proximal and distal ends of the housing, wherein the adapter is coupled with the insertion shaft of the first endoscope at a distal end thereof and comprises a port at a proximal end thereof that is configured to reside within an opening defined by the proximal end of the housing and define a proximal entryway into the instrument channel.
Example 89. The system of Example 72 or Example 88, wherein the second endoscope further includes an adapter within the housing of the second endoscope that extends between the proximal and distal ends of the housing, wherein the second adapter is coupled with the insertion shaft of the second endoscope at a distal end thereof and comprises a plug at a proximal end thereof that is configured to reside within an opening defined by the proximal end of the housing to close the opening.
Example 90. The system of any one of Example 87 to Example 89, wherein each housing of the first and second endoscopes is configured to pivotally retain the actuator of the respective first or second endoscope.
Example 91. An endoscope comprising:
a housing elongated along a longitudinal axis between a proximal end and a distal end, the housing defining a proximal opening at the proximal end and a distal opening at the distal end;
an adapter coupled with the housing, the adapter comprising a distal end positioned within the distal opening of the housing and further comprising a proximal end positioned within the proximal opening of the housing; and
an elongated insertion shaft connected to the adapter and extending distally away from the distal opening of the housing.
Example 92. The endoscope of Example 91, wherein the proximal end of the adapter comprises a port that defines a proximal end of an instrument channel.
Example 93. The endoscope of Example 92, wherein the instrument channel extends through the housing and through a full length of the insertion shaft.
Example 94. The endoscope of any one of Example 91 to Example 93, further comprising a valve assembly coupled with the adapter.
Example 95. The endoscope of any one of Example 92 to Example 94, wherein the valve assembly defines a selectively sealable portion of the instrument channel.
Example 96. The endoscope of Example 91, further comprising a fluid port, wherein the adapter comprises a fluid aperture coupled with the fluid port.
Example 97. The endoscope of Example 96, wherein the fluid aperture is coupled with a channeling tube that extends through the insertion shaft, the channeling tube being configured to conduct fluid distally through the insertion shaft after the fluid has entered the endoscope at the fluid port.
Example 98. The endoscope of Example 97, wherein the channeling tube extends through the fluid aperture of the adapter.
Example 99. The endoscope of Example 97, wherein the channeling tube defines a portion of an instrument channel through which an elongated instrument can be advanced distally through the insertion shaft.
Example 100. The endoscope of Example 91, wherein the proximal end of the adapter comprises a plug that closes the proximal opening of the housing.
Example 101. A system comprising:
a handle comprising:
   a housing configured to be coupled with an endoscope, the housing defining an interior space;
   an inductive coil within the interior space of the housing;
   a rechargeable battery within the interior space of the housing and electrically coupled with the inductive coil; and
   an electrical connector coupled with the housing via a fluid-tight seal, the electrical connector comprising an opening and a recess that extends inwardly into the interior space of the housing; and
a charging cradle comprising:
   a housing that comprises a base and defines an external receptacle configured to receive the handle; and
   an inductive coil positioned within the housing,
wherein, when the charging cradle rests on the base of its housing and the handle is received in the receptacle of the charging cradle housing, the inductive coils of the handle and the charging cradle are oriented to permit inductive charging of the rechargeable battery and the opening of the electrical connector is oriented lower than the recess of the electrical connector to permit cleaning fluid to egress from the electrical connector under the influence of gravity.
Example 102. The system of Example 101, wherein each inductive coil extends about a separate coiling axis that is normal to a coiling plane, and wherein the coiling plane of each inductive coil is substantially vertical when the charging cradle rests on the base of its housing and the handle is received in the receptacle of the charging cradle housing.
Example 103. The system of Example 101 or Example 102, wherein the opening of the electrical connector faces downward when the charging cradle rests on the base of its housing and the handle is received in the receptacle of the charging cradle housing.
Example 104. The system of any one of Example 101 to Example 103, wherein the opening of the electrical connector is vertically beneath the recess of the electrical connector when the charging cradle rests on the base of its housing and the handle is received in the receptacle of the charging cradle housing.
Example 105. The system of any one of Example 101 to Example 104, wherein the handle comprises a convex surface and the cradle comprises a concave surface sized to receive the convex surface of the handle.
Example 106. The system of any one of Example 101 to Example 105, wherein the receptacle of the charging cradle comprises one or more vertically oriented ribs configured to press a base surface of the housing of the handle against a vertical sidewall of the receptacle.
Example 107. The system of Example 106, wherein the inductive coil of the handle is positioned flatly adjacent to the base surface of the housing and the inductive coil of the charging cradle is positioned flatly adjacent to the vertical sidewall of the receptacle.

Any suitable combination of the various features of the various embodiments and examples disclosed herein is contemplated. The term “coupled to” can mean connected to in any suitable fashion, whether that coupling is direct or indirect. Separate components may be coupled to each other. Moreover, in some instances, where separately identified components are integrally formed from a unitary piece of material, or stated otherwise, are included together in a monolithic element, those elements may also be said to be coupled to one another.

Although the foregoing detailed description contains many specifics for the purpose of illustration, a person of ordinary skill in the art will appreciate that many variations and alterations to the following details can be made and are considered to be included herein. Accordingly, the foregoing embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any one of claims [x] through the immediately preceding claim,” where the bracketed term “[x]” is replaced with the number of the most recently recited independent claim. For example, for the first claim set that begins with independent claim 1, claim 3 can depend from either of claims 1 and 2, with these separate dependencies yielding two distinct embodiments; claim 4 can depend from any one of claim 1, 2, or 3, with these separate dependencies yielding three distinct embodiments; claim 5 can depend from any one of claim 1, 2, 3, or 4, with these separate dependencies yielding four distinct embodiments; and so on.

Recitation in the claims of the term “first” with respect to a feature or element does not necessarily imply the existence of a second or additional such feature or element.

1. An endoscope comprising:
a handle;
an actuator coupled to the handle;
an elongated shaft coupled to the handle, the shaft comprising:
a deflectable distal portion;
an imaging assembly at a distal end of the shaft;
a first tube that defines a hollow interior; and
an elongated guide member positioned within the interior of the first tube, the guide member comprising:
a solid interior such that a transverse cross section of the guide member along at least a portion of a full longitudinal length of the guide member is devoid of any internal openings;
a first longitudinal groove at an external surface of the guide member that cooperates with the first tube to define a first lumen; and
a second longitudinal groove at the external surface of the guide member that cooperates with the first tube to define a second lumen through which communication lines associated with the imaging assembly pass; and
a steering wire coupled to the deflectable distal portion of the shaft, extending through the first lumen, and coupled with the actuator such that actuation of the actuator causes the steering wire to deflect the distal portion of the shaft.

2. The endoscope of claim 1, wherein the guide member comprises a third longitudinal groove at the external surface of the guide member that cooperates with the first tube to define a third lumen through which a second tube passes.

3. The endoscope of claim 2, wherein the handle comprises a fluid port coupled with the second tube, the fluid port being configured to deliver fluid into the second tube for advancement of the fluid toward the distal end of the shaft through the third lumen.

4. The endoscope of claim 2, wherein the handle comprises an instrument port coupled with the second tube, the instrument port being configured to receive an elongated instrument therethrough and into the second tube for advancement of the instrument toward the distal end of the shaft through the third lumen.

5. The endoscope of any one of claim 2, wherein the guide member further comprises a fourth longitudinal groove at the external surface of the guide member that cooperates with the first tube to define a fourth lumen through which an additional steering wire extends.

6. The endoscope of claim 1, wherein the guide member comprises an extrusion of a polymeric material.

7. The endoscope of claim 1, wherein a central longitudinal axis of the first tube extends through the guide member.

8. The endoscope of claim 1, wherein the first tube comprises a rigid portion that extends proximally from the deflectable distal portion of the shaft.

9. The endoscope of claim 8, wherein the first tube comprises a bending section that corresponds with at least a portion of the deflectable distal portion of the shaft.

10. The endoscope of claim 9, wherein the bending section of the first tube comprises a plurality of laser-cut grooves.

11. The endoscope of claim 9, wherein the guide member comprises a distal tip that terminates at a position at or proximal to a proximal end of the bending section.

12. The endoscope of claim 9, wherein the guide member extends through at least a portion of each of the rigid portion and the bending section.

13. The endoscope of claim 1, wherein the handle is separable into a reusable portion and a disposable portion.

14. The endoscope of claim 13, wherein the reusable portion comprises circuitry for wireless communication with a processor and/or a display monitor.

15. The endoscope of claim 13, wherein the disposable portion comprises the actuator.

16. The endoscope of claim 1, wherein the handle defines a channel through which elongated instruments may be passed.

17. The endoscope of claim 16, wherein the endoscope further comprises a working channel, and wherein the channel defined by the handle constitutes a proximal end of the working channel.

18. The endoscope of claim 17, wherein a portion of the working channel is defined by a separate accessory that is selectively attachable to and detachable from the handle.

19. An endoscope comprising:
a tube comprising an inner surface that defines a primary lumen;
first and second steering wires positioned within the primary lumen of the tube; and
a guide member positioned within the primary lumen of the tube, the guide member comprising:
an elongated body that defines an outer perimeter, the body being solid such that an entirety of a region internal to the outer perimeter is devoid of any passageways;
a plurality of ribs extending longitudinally along the body to define at least a portion of the outer perimeter of the body;
a first steering lumen positioned angularly between a first pair of the plurality of ribs, the first steering lumen being defined by a first portion of the outer perimeter of the elongated body and a first portion of the inner surface of the tube, the first steering wire extending longitudinally through the first steering lumen; and
a second steering lumen positioned angularly between a second pair of the plurality of ribs, the second steering lumen being defined by a second portion of the outer perimeter of the elongated body and a second portion of the inner surface of the tube, the second steering wire extending longitudinally through the second steering lumen.

20. An endoscope comprising:
a first tube comprising an inner surface that defines a primary lumen;
a channeling tube positioned within the primary lumen of the tube;
first and second steering wires positioned within the primary lumen of the tube; and
a guide member positioned within the primary lumen of the tube, the guide member comprising:
an elongated body that defines an outer perimeter;
a plurality of ribs extending longitudinally along the body to define at least a portion of the outer perimeter of the body;
a first steering lumen positioned angularly between a first pair of the plurality of ribs, the first steering lumen being defined by a first portion of the outer perimeter of the elongated body and a first portion of the inner surface of the tube, the first steering wire extending longitudinally through the first steering lumen;
a second steering lumen positioned angularly between a second pair of the plurality of ribs, the second steering lumen being defined by a second portion of the outer perimeter of the elongated body and a second portion of the inner surface of the tube, the second steering wire extending longitudinally through the second steering lumen; and
a channeling lumen positioned angularly between a third pair of the plurality of ribs, the channeling lumen being defined by a third portion of the outer perimeter of the elongated body and a third portion of the inner surface of the tube, the channeling tube extending longitudinally through the channeling lumen.
